(19) United States
(12) Patent Application Publication
(10) Pub. No.: US 2026/0071275 A1
(43) Pub. Date: Mar. 12, 2026

(54) USE OF C9ORF72 -MEDIATED GENES FOR DIAGNOSIS AND TREATMENT OF NEURONAL DISEASES

(71) Applicant: SELONTERRA, INC., SAN MATEO, CA (US)

(72) Inventors: Anne URFER-BUCHWALDER, Belmont, CA (US); Roman URFER, Belmont, CA (US)

(73) Assignee: SELONTERRA, INC., SAN MATEO, CA (US)

(21) Appl. No.: 18/697,487

(22) PCT Filed: Sep. 23, 2022
(86) PCT No.: PCT/US2022/044464

§ 371 Date: Apr. 1, 2024

Related U.S. Application Data
(60) Provisional application No. 63/250,519, filed on Sep. 30, 2021.

(51) Int. Cl. C12Q 1/6883; C12Q 1/6813

(52) U.S. Cl. CPC C12Q 1/6883; C12Q 1/6813; C12Q 2600/158

(57) ABSTRACT

The present disclosure provides compositions and methods using C9ORF72-mediated genes and expression products thereof for diagnosis, treatment and prevention of amyotrophic lateral sclerosis, frontotemporal dementia, or both, in carriers of a C9ORF72 hexanucleotide expansion. The present invention also relates to a method of identifying therapeutic agents to treat and diagnose amyotrophic lateral sclerosis, frontotemporal dementia, or both, in carriers of a C9ORF72 hexanucleotide expansion based on C9ORF72-mediated genes.

### CLAIM OF PRIORITY

This application claims priority to U.S. Application Ser. No. 63/250,519, filed Sep. 30, 2021, the contents of which are herein fully incorporated by reference in its entirety.

### INCORPORATION BY REFERENCE STATEMENT

This application incorporates by reference the sequence listing having the file name 03032SI08USNAT4US-2025-11-17.xml created Nov. 17, 2025 with a file size of 2,079 bytes and having been filed in the United States and Trademark Office via EFS Web on Nov. 17, 2025.

### FIELD OF THE INVENTION

The present invention relates to a method of diagnosing, preventing, and treating neuronal diseases in carriers of C9ORF72 (chromosome 9 open reading frame 72) hexanucleotide expansions. Methods of the invention are based at least in part on measuring or modulating one or more genes, or their expression products, from a set of genes (referred to herein as the “C9ORF72-mediated genes”) identified by the present invention as being involved in the etiology of amyotrophic lateral sclerosis (ALS) or frontotemporal dementia (FTD) in carriers of C9ORF72 hexanucleotide expansions.

### BACKGROUND OF THE INVENTION

C9ORF72 is a gene located on human chromosome 9 and codes for an open reading frame of unclear activity. The C9ORF72 gene can carry hexanucleotide expansions of various length in the intron located between exon 1a and 1b. These hexanucleotide expansions are genetic variants in the human genome and were associated with amyotrophic lateral sclerosis (ALS) and frontotemporal dementia (FTD) in carriers of these genetic variants.

ALS affects people worldwide. Currently, however, there are no reliable and effective methods for treatment or prevention of ALS. It is believed that ALS is caused by a combination of environmental, age and genetic factors. ALS consists of idiopathic and familial (inherited) forms. The underlying neurodegeneration in ALS often goes unrecognized in its very early stages where treatments might be most effective. Current Food and Drug Administration (FDA) approved ALS drugs have only modest effects on improving the patient's daily functioning but do not slow down the disease process or treat the underlying pathology.

FTD occurs in patients worldwide. Currently, however, there are no reliable and effective methods for treatment or prevention of FTD. It is believed that FTD is caused by a combination of environmental, age and genetic factors. FTD is mostly a familial (inherited) disease. The underlying neurodegeneration in FTD often goes unrecognized in its very early stages where treatments might be most effective. There are currently no Food and Drug Administration (FDA) approved drugs for the specific treatment of FTD and current treatments provide only some symptomatic management. There is therefore a large unmet medical need for drugs improving the patient's daily functioning, slowing down the disease process or treating the underlying pathology.

C9ORF72 hexanucleotide expansions provide for a common genetic association for ALS and FTD.

The biochemical basis of the very tissue specific degeneration of neurons in ALS and FTD in carriers of the C9ORF72 hexanucleotide expansions is not fully understood.

Unfortunately, it is not clear whether the biochemical events observed are a cause or a result of the development of ALS and FTD. The conventional research apparently does not relate to the actual mechanism of causation of ALS and FTD in carriers of the C9ORF72 hexanucleotide expansions but rather only relates to the consequences of the underlying mechanism that causes ALS or FTD. If one can gain detailed knowledge of the underlying mechanism for development of ALS and FTD in carriers of C9ORF72 hexanucleotide expansions, it would be possible to accurately diagnose, treat and/or prevent development of ALS and FTD in these subjects.

One tantalizing clue towards possible treatments for ALS or FTD in carriers of C9ORF72 hexanucleotide expansions comes from genetic research which revealed that people carrying specific alleles of C9ORF72 are at substantially increased risk of developing ALS or FTD, or both. The C9ORF72 hexanucleotide expansions were the first described genetic link between familial FTD and ALS. This genetic risk of C9ORF72 alleles extends to both the idiopathic and familial forms of ALS. This genetic discovery has triggered substantial investigation into the role C9ORF72 and its different alleles may play in the development of ALS and FTD. To date there is no generally accepted explanation for the correlation between C9ORF72 activity, which occurs in many regions of the brain, and the observed pathological effects of ALS or FTD which are limited to very specific areas of the brain, the motor neurons in the former and the neurons of the frontal and temporal lobes in the latter. Here again, this research area has been frustrating and has not yet led to new treatments for ALS or FTD in carriers of C9ORF72 hexanucleotide expansions.

Since there is no treatment available for carriers of C9ORF72 hexanucleotide expansions that develop ALS or FTD, or both, that address the underlying cause of these disease in these carriers, there is still a need for a method for treating these carriers by treating the actual cause of ALS and FTD in these carriers.

### SUMMARY OF THE INVENTION

Some aspects of the present invention are based on the discovery of a set of genes that are directly responsible for the development of ALS or FTD in carriers of C9ORF72 hexanucleotide expansions. The present invention provides multiple new avenues for diagnosing, preventing and/or treating ALS and FTD in these subjects.

The human C9ORF72 gene has variant alleles that carry an insert of hexanucleotides of various lengths in the intron located between the exon 1a and 1b of the C9ORF72 gene. One single hexanucleotide has the sequence GGGGCC (SEQ 1A) and these hexanucleotides occur in multiples of this sequence to form long repeated sequences inserted into the C9ORF72 gene. The number of hexanucleotides inserted determines the absence or presence of ALS or FTD, or both, in carriers of these variant alleles. Healthy individuals have 23 hexanucleotide repeats or less while a disease allele size of 24 and more hexanucleotide repeats has been identified. Other researchers reported hexanucleotide repeats associated with disease in the hundreds of hexanucleotide repeats. Therefore, a C9ORF72 hexanucleotide expansion allele is defined as a C9ORF72 allele with preferably at least 24 hexanucleotide repeats, more preferably at least 50 hexanucleotide repeats and most preferably at least 100 hexanucleotide repeats. The C9ORF72 wild-type is defined as a C9ORF72 allele with less than 24 hexanucleotide repeats.

A carrier of a C9ORF72 hexanucleotide expansion is defined as a subject carrying a C9ORF72 allele with preferably at least 24 hexanucleotide repeats, more preferably at least 50 hexanucleotide repeats and most preferably at least 100 hexanucleotide repeats. The subject can be homozygous or heterozygous for the C9ORF72 hexanucleotide expansion allele.

C9ORF72 hexanucleotide expansions have been identified in numerous families with heritable ALS or FTD. C9ORF72 hexanucleotide expansions have also been associated with idiopathic cases of ALS.

The present invention recognized that the effects of C9ORF72 hexanucleotide expansions on the functions of the C9ORF72 gene itself and its encoded expression products do not explain important aspects of the link of C9ORF72 genomic variants to ALS and FTD in carriers of C9ORF72 hexanucleotide expansions. While ALS and FTD manifest themselves primarily in a degeneration of motor neurons and neurons of the frontal and temporal lobes, respectively, the expression products of the C9ORF72 gene can be detected in many regions of the brain.

The present invention reveals that the effects on the causation of ALS or FTD, or both, by a C9ORF72 hexanucleotide expansion is not due to the expression products of the C9ORF72 gene itself but rather due to the altered expression of genes in the vicinity of the C9ORF72 gene when the allele is a C9ORF72 hexanucleotide expansion. The expression of these genes is altered by the insertion of hexanucleotide repeats into the C9ORF72 gene.

One particular aspect of the invention is based on the analysis of the gene structure of C9ORF72 hexanucleotide expansions and the presence of genes in the vicinity of the C9ORF72 gene.

The present invention demonstrates effects in the presence of a C9ORF72 hexanucleotide expansion on the transcription of genes, the “C9ORF72-mediated genes”, in the chromosomal vicinity of the C9ORF72 gene, within about 2 Mb upstream or downstream of the location of the C9ORF72 gene on human chromosome 9.

One particular aspect of the invention is based on the analysis of the gene structure of C9ORF72 hexanucleotide expansions and the unexpected discovery that these C9ORF72 hexanucleotide expansions act at their DNA level and contain recognition motifs for transcription factors SP1, KLF5, ZFX, KLF4, SP2, EGR1, KLF16 and TFAP2A.

These remarkable findings by the present invention reveal for the first time that C9ORF72 hexanucleotide expansions are unexpectedly modulating (e.g., activating or suppressing) transcription of genes located in the genomic vicinity of C9ORF72. These C9ORF72-mediated genes are not activated (or suppressed) to the same extent by the C9ORF72 wild-type, thereby providing an explanation for the correlation of the presence of C9ORF72 hexanucleotide expansions and ALS or FTD in carriers of a C9ORF72 hexanucleotide expansion. As discussed herein, C9ORF72-mediated gene expression can result in inhibition or activation of a particular C9ORF72-mediated gene or the activity of the gene expression product. Accordingly, throughout this disclosure, it should be understood that when the term “modulate” is used in reference to C9ORF72-mediated gene expression or the gene expression product thereof, the term “modulate” can alternatively be substituted with the term “increase”, “activate”, “decrease” or “inhibit”. For example, if a particular gene expression (or the activity of the gene expression product thereof) is decreased due to the presence of a C9ORF72 hexanucleotide expansion, then the methods of the invention will be directed to increasing the gene expression (or the activity of the gene expression product thereof). Similarly, if a particular gene expression (or the activity of the gene expression product thereof) is increased due to the presence of a C9ORF72 hexanucleotide expansion, then the methods of the invention will be directed to inhibiting the gene expression (or the activity of the gene expression product thereof).

One particular aspect of the invention provides a method of modulating (i.e., increasing or decreasing) the C9ORF72 hexanucleotide expansion-mediated (i.e., regulated) expression of a gene or the activity of gene product thereof. In some embodiments, modulation of the expression of a C9ORF72-mediated gene is achieved by contacting a cell that is expressing a gene mediated by a C9ORF72 hexanucleotide expansion with a molecule. As used herein unless the context requires otherwise, the terms “molecule” and “compound” are used interchangeably herein and refers to any molecule known to one skilled in the art, such as, but not limited to, small molecules, oligonucleotides (including short interfering RNAs and aptamers), peptides, polypeptides (including aptamers, zinc fingers and fragments thereof), proteins (including antibodies and fragments thereof) as well as derivatives or modified forms thereof. In some embodiments, a molecule is delivered using a viral vector. In some embodiments, the cell is a neuronal cell, neuronal progenitor cell, differentiated neuron, oligodendrocyte, fibroblast, lymphocyte, human embryonic kidney cell or another cell type or extracts thereof. In other embodiments, the modulation of the activity of a C9ORF72-mediated gene expression product is achieved by contacting said gene expression product with a molecule that is capable of selectively modulating the activity of said gene expression product.

Yet in other embodiments, the C9ORF72-mediated gene is located on human chromosome 9 within about 2 Mb upstream or downstream of the location of C9ORF72 (ENSG00000147894; chromosomal location of C9ORF72 9:27535640-27573866). In other embodiments, the C9ORF72-mediated gene is selected from the group consisting of IZUMO3, TUSC1, CAAP1, PLAA, IFT74, LRRC19, TEK, EQTN, MOB3B, IFNK and LINGO2 or a combination thereof. In a further embodiment, the C9ORF72-mediated gene comprises PLAA, MOB3B and TEK or a combination thereof.

In one particular embodiment, the expression or the activity of a C9ORF72-mediated gene is modulated using a molecule or a compound. As used herein, the terms “C9ORF72-mediated gene,” “gene mediated by a C9ORF72 hexanucleotide expansion” are used interchangeably herein and refer to gene(s) located on human chromosome 9 within about 5 Mb, typically within about 4 Mb, often within about 3 Mb, and most often within about 2 Mb upstream or downstream of the location of C9ORF72 (ENSG00000147894; chromosomal location of C9ORF72 9:27535640-27573866). In one particular embodiment, the C9ORF72-mediated gene is selected from the group consisting of IZUMO3, TUSC1, CAAP1, PLAA, IFT74, LRRC19, TEK, EQTN, MOB3B, IFNK and LINGO2 or a combination thereof. In a further embodiment, the C9ORF72-mediated gene comprises PLAA, MOB3B and TEK or a combination thereof. For additional clarity, a C9ORF72-mediated gene is one with sufficient chromosomal proximity to the C9ORF72 gene that its expression level is modified by the presence of a C9ORF72 hexanucleotide expansion.

Such C9ORF72-mediated genes have a standard expression or activity level based on the C9ORF72 wild-type (non-disease causing) allele. A C9ORF72 hexanucleotide expansion causes significantly different expression levels of C9ORF72-mediated genes or significantly different activity levels of expression products thereof, thereby putting a carrier of a C9ORF72 hexanucleotide expansion at risk of ALS, FTD or both. The terms “about” and “approximately”, when used to describe a numeric value, are used interchangeably herein and are not intended to limit the scope of the invention unless indicated otherwise. The terms “about” and “approximately” refer to being within an acceptable error range for the particular value as determined by one of ordinary skill in the art, which will depend in part on how the value is measured or determined, i.e., the limitations of the measurement system, i.e., the degree of precision required for a particular purpose. For example, the terms “about” and “approximately” can mean within 1 or more than 1 standard deviation, per the practice in the art. Alternatively, the terms “about” and “approximately” when referring to a numerical value can mean±20%, typically ±10%, often ±5% and more often ±1% of the numerical value. In general, however, where particular values are described in the application and claims, unless otherwise stated, the terms “about” and “approximately” mean within an acceptable error range for the particular value. The term “modulate” when used in reference to C9ORF72-mediated gene expression includes reducing or increasing transcription and/or translation of the C9ORF72-mediated gene. This can include downregulating or complete suppression or upregulating of the expression products of a C9ORF72-mediated gene as compared to a control (e.g., in the absence of a molecule). The term “modulate” when used in reference to the activity of a C9ORF72-mediated gene expression product includes reducing or increasing the activity of the C9ORF72-mediated gene. Typically, methods of the invention show at least about 25%, typically at least about 50%, and often at least about 75% modulation of C9ORF72-mediated gene expression or activity of the expression product thereof.

This method can be used inter alia to identify a lead candidate for a drug development for treatment of ALS, FTD or both, in a carrier of a C9ORF72 hexanucleotide expansion.

The molecule used for modulating the expression or activity of a C9ORF72-mediated gene includes, but is not limited to, small molecules, oligonucleotides (including short interfering RNAs, RNAs and aptamers), peptides, polypetides (including aptamers, zinc fingers and fragments thereof), proteins (including antibodies and fragments thereof) as well as derivatives or modified forms thereof.

In some instances, the oligonucleotide is 9 to 30 nucleotides in length comprising consecutive nucleotide sequences within SEQ ID NO:1. In some cases, the oligonucleotide is a single-stranded oligonucleotide while in other instances the oligonucleotide is a double-stranded oligonucleotide. The oligonucleotide can also be a phosphorothioate oligonucleotide, a methylphosphonate oligonucleotide, a phosphoamidite oligonucleotide, a peptide nucleic acid oligonucleotide, a locked-nucleic acid-modified oligonucleotide, or combinations thereof.

The oligonucleotide sequence of SEQ ID NO: 1 is GGGGCCGGGGCCGGGGCCGGGGCCGGGGCC corresponding to a C9ORF72 hexanucleotide expansion with five repeated elements.

In some embodiments, the molecule further comprises a pharmaceutically acceptable carrier.

In some embodiments, the molecule is delivered by a viral vector.

Another aspect of the invention provides a method for identifying a molecule that can modulate the binding of a transcription factor to the C9ORF72 hexanucleotide expansion. This method can be used inter alia to identify a lead candidate for a drug development for treatment of ALS, FTD, or both, in a carrier of a C9ORF72 hexanucleotide expansion.

Yet another aspect of the invention provides a method for modulating (i.e., increasing or decreasing) the expression or activity of a C9ORF72-mediated gene or expression products thereof in a cell. The method comprises contacting a cell that is expressing a C9ORF72-mediated gene with a molecule that is capable of modulation the expression or the activity of said gene. This method can be used inter alia to identify a lead candidate for a drug development for treatment of ALS, FTD or both, in a carrier of a C9ORF72 hexanucleotide expansion. Yet in other embodiments, the molecule includes, but is not limited to, small molecules, oligonucleotides (including short interfering RNAs, RNAs and aptamers), peptides, polypetides (including aptamers, zinc fingers and fragments thereof), proteins (including antibodies and fragments thereof) as well as derivatives or modified forms thereof.

Yet another aspect of the invention provides a method for treating a subject who is a carrier of a C9ORF72 hexanucleotide expansion for ALS, FTD, or both. The method includes determining the presence of a C9ORF72 hexanucleotide expansion in the subject; and (a) if said subject carries a C9ORF72 hexanucleotide expansion, administering said subject with a molecule that is capable of modulating the expression or activity of a C9ORF72-mediated gene; or (b) if said subject does not carry C9ORF72 hexanucleotide expansion, administering said subject with a molecule that is different from said molecule of (a). In some embodiments, the method can include a step of determining whether said subject is homozygous or heterozygous for the C9ORF72 hexanucleotide expansion. As used herein, the term “treating” or “treatment” of a disease includes: (1) preventing the disease, i.e., causing the clinical symptoms of the disease not to develop in a mammal that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

The advantages of the present invention include a transformative approach to treatment of carriers of a C9ORF72 hexanucleotide expansion for ALS, FTD, or both, targeted therapies addressing dysfunction caused by C9ORF72-mediated genes that delay onset of ALS, FTD or both, prevent their progression or reverse their symptoms, and provide disease-modifying therapies, i.e., treating the actual cause of ALS, FTD, or both, rather than treating mere symptoms of ALS, FTD. or both, in carriers of a C9ORF72 hexanucleotide expansion.

### BRIEF DESCRIPTION OF THE DRAWINGS

FIG. 1: Effect of the C9ORF72 hexanucleotide expansion on the transcription of C9ORF72-mediated genes. FIG. 1A shows the C9ORF72 wild-type allele with C9ORF72-mediated genes in the vicinity of C9ORF72. FIG. 1B shows inhibition of expression of a C9ORF72-mediated gene by the presence of a C9ORF72 hexanucleotide expansion. FIG. 1C shows increased expression of a C9ORF72-mediated gene by the presence of a C9ORF72 hexanucleotide expansion.

FIG. 2: Inhibition of YAP1 transcriptional activity by the Hippo pathway. Role of C9ORF72-mediated genes MOB3B and PLAA in the YAP1/Hippo pathway is shown.

FIG. 3: Mechanism of activation of YAP1 transcriptional activity. Role of C9ORF72-mediated gene TEK in the YAP1 transcriptional activation pathway is shown.

### DETAILED DESCRIPTION OF THE INVENTION

Amyotrophic lateral sclerosis (ALS) is a motor neuron disease that affects upper and lower motor neurons with neuronal death occurring in the brain, brain stem and spinal cord. The disease occurs usually in middle age and the course of disease is generally fatal within 3 to 5 years after the initial diagnosis. The disease presents itself as a progressive muscle atrophy and weakness, and the effects on respiratory muscles lead to respiratory failure and death.

Current treatment options are extremely limited and include respiratory support and the management of symptoms. The two approved therapies, Riluzole and Edaravone, provide only modest benefits and therefore, a significant unmet medical need exists.

There are several forms of the disease including sporadic and familial forms as well as a juvenile variant of ALS. ALS is the most common motor neuron disease and is an orphan disease with an incidence of 1 to 2 per 100,000 subjects and a prevalence of about 5 cases per 100,000 individuals.

Familial forms of ALS contribute to about 10% of ALS cases and are transmitted in families almost always as a dominant trait with high penetrance. These include SOD1, DCTN1, ANG, TARDBP, FUS, VCP, OPTN, C9ORF72, UBQLN2, SQSTM1, PFN1, HNRNPA1, MATR3, TUBA4A, CHCHD10, TBK1ⁱ. Another review of ALS genes listed TARDBP, FUS, ANG, SETX, MATR3, ATXN2, TAF15, EWSR1, hnRNPal, hnRNPA2B1, ELP3, C9ORF72, ALS2, VAPB, CHMP2B, FIG. 4, UBQLN2, SQSTM1, SIGMAR1, OPTN, VCP, TBK1, DCTN1, PFN1, SPG11, TUBA4A, NEFH, PRPH, NEK1, CHCHD10, C19ORF12, SOD1, ERBB4, SS18L1, PNPLA6, PON1-3, DAO, CHRNA3, CHRNA4, CHRNB4, ALS3, ALS7, ALS6-21 and ALS-FTDⁱⁱ.

The most prevalent ALS gene in familial forms is C9ORF72 displaying hexanucleotide expansions with an occurrence in approximately 40% of cases. Sporadic forms of ALS often carry genetic variants known from the study of the familial forms. For example, about 1-3% of sporadic cases carry missense mutations in SOD1ⁱⁱⁱ and another 8-10% are caused by C9ORF72 hexanucleotide expansionsiv. C9ORF72 hexanucleotide expansions are the most common genetic variant associated with ALS.

Frontotemporal dementia (FTD) is a pre-senile dementia in which degeneration of the frontal and temporal lobes of the brain results in progressive executive, behavior, and language dysfunctions^{v}. FTD is the most common dementia in people aged under 60 years^{vi}. As many as half of the FTD patients develop motor neuron dysfunction^{vii}.

The only approved drug for any form of FTD is riluzole and many other off-label used drugs are targeted at the management of neuropsychiatric symptoms. None of these approaches target the pathological processes of FTD^{viii}. Therefore, a significant unmet medical need exists for the therapy of FTD patients.

Genetic contributions play an important role in FTD. Approximately 30% of patients with FTD have a family history of dementia. About 60% of patients with inherited FTD have autosomal dominant mutations in the C9ORF72, GRN or MAPT genes^{ix}. Further rare variants associated with FTD have been identified in the genes VCP, CHM2B, TARDBP, FUS, UBQLN2, SQSTM1, CHCHD10, TBK1, OPTN, CCNF and TIA1^{viii}.

The human C9ORF72 gene has been linked to both ALS and FTD^{x,xi}.

The human C9ORF72 gene has variant alleles that carry an insert of hexanucleotides of various lengths in the intron located between the exon 1a and 1b of the C9ORF72 gene. One single hexanucleotide has the sequence GGGGCC (SEQ 1A) and these hexanucleotides occur in multiples of this sequence to form long repeated sequences inserted into the C9ORF72 gene. The number of hexanucleotides inserted determines the absence or presence of ALS or FTD, or both, in carriers of these variant alleles. Healthy individuals have 23 hexanucleotide repeats or less while a disease allele size of 24 and more hexanucleotide repeats has been identified. Other researchers reported hexanucleotide repeats associated with disease in the hundreds of hexanucleotide repeats. Therefore, a “C9ORF72 hexanucleotide expansion” is defined as a C9ORF72 allele with preferably at least 24 hexanucleotide repeats, more preferably at least 50 hexanucleotide repeats and most preferably at least 100 hexanucleotide repeats. The C9ORF72 wild-type is defined as a C9ORF72 allele with less than 24 hexanucleotide repeats.

A carrier of a C9ORF72 hexanucleotide expansion is defined as a subject carrying a C9ORF72 allele with preferably at least 24 hexanucleotide repeats, more preferably at least 50 hexanucleotide repeats and most preferably at least 100 hexanucleotide repeats. The subject can be homozygous or heterozygous for the C9ORF72 hexanucleotide expansion allele.

There is uncertainty about the mechanism by which C9ORF72 causes ALS and/or FTD and proposed mechanisms include the formation of toxic RNA species forming RNA nuclei, a dysfunction of its protein activity as a guanine nucleotide exchange factor and the formation of toxic dinucleotide repeats encoded by the hexanucleotide insertion into the C9ORF72 gene.

Despite significant efforts to identify therapies addressing these C9ORF72 hexanucleotide expansions no therapy addressing this dominant genetic cause has been approved for the treatment of ALS or FTD patients.

Reduced penetrance, variability in expression both in tissues and subjects, and striking pathological differences within mutations affecting the same protein/protein domain are difficult to explain with a faulty protein function mechanism.

The present invention demonstrates that C9ORF72 hexanucleotide expansions cause ALS, FTD, or both, through an alternative, DNA based mechanism that allows incorporation of exposure to external factors in carriers of C9ORF72 hexanucleotide expansions. Tissue specific gene expression is controlled by DNA sequences called cis-regulatory modules that can function over large genomic distances. Transcription factors and other DNA binding proteins bind to these regulatory elements resulting in stimulation/enhancement or alternatively repression of gene transcription. The invention demonstrates that C9ORF72 hexanucleotide expansions either create new enhancer elements via de novo transcription factor binding sites, or alter existing enhancers that control the expression of a spatially close neighboring gene, the “C9ORF72-mediated gene”.

These remarkable findings by the present invention reveal that C9ORF72 hexanucleotide expansions are unexpectedly modulating (e.g., activating or suppressing) transcription of a range of genes in the vicinity of C9ORF72. These genes, located in the genomic vicinity of C9ORF72, excluding C9ORF72, are herein referred to as C9ORF72-mediated genes. These genes are not activated (or suppressed) to the same extent by the C9ORF72 wild-type, thereby for the first time providing an alternative explanation for the correlation of C9ORF72 hexanucleotide expansions with ALS and FTD.

This DNA based mechanism allows for the incorporation of exposure to external factors in carriers of C9ORF72 hexanucleotide expansions.

The most common means of transcriptional regulation occurs in cis, as 98% of chromatin loops anchored at a promoter are located within a range of 2 Mb of the enhancer's location, indicating that the vast majority of genes regulated by the enhancer are located within 2 Mb of the enhancer's chromosomal positionNii. Studies from a variety of organisms suggest that mutations affecting cis-regulatory activity are the predominant source of expression divergence between species.

Transcriptional regulation emanating from enhancer elements can also function in trans, meaning that they can regulate the expression of genes on chromosomes distinct from the chromosome of the transcriptional enhancer element.

In some embodiments, “C9ORF72-mediated gene” means any gene located within 2 Mb (2 megabases=2 million bases) upstream or downstream of the location of C9ORF72 (ENSG00000147894; chromosomal location of C9ORF72 9:27535640-27573866). Still in other embodiments, the term “C9ORF72-mediated gene” or the “expression product” refers to the gene or the expression product, respectively, that is modulated (e.g., either increased or decreased) by the presence of C9ORF72 hexanucleotide expansions. In some cases, the activity of the C9ORF72-mediated gene or the expression product thereof is increased in C90RF72 hexanucleotide expansions compared to C9ORF72 wild-type. In other cases, the activity of the gene or the expression product thereof is reduced in C9ORF72 hexanucleotide expansions compared to C9ORF72 wild-type. A transcription product of such a gene includes any RNA transcript based on such a gene, including any microRNA or mRNA (whether the mRNA transcript is primary, spliced, edited, modified or mature). A “C9ORF72-mediated gene expression product” as used herein means a polypeptide translated from an mRNA transcript of an C9ORF72-mediated gene, any mRNA transcript including but not limited to mRNA (whether the mRNA transcript is primary, spliced, edited, modified or mature) or a microRNA. A polypeptide may be nascent or processed into a mature or modified form of the protein.

A representative list of the C9ORF72-mediated genes, including genes located on chromosome 9 in about a 2 Mb window upstream or downstream of the location of C9ORF72 (ENSG00000147894; chromosomal location of C9ORF72 9:27535640-27573866), was compiled from the GRCh38.p13 Homo sapiens Genome Assembly of EnsEmbl and is provided in Table 1. These genes include IZUMO3, TUSC1, CAAP1, PLAA, IFT74, LRRC19, TEK, EQTN, MOB3B, IFNK and LINGO2. Still in other embodiments, the C9ORF72-mediated genes comprise IZUMO3, TUSC1, CAAP1, PLAA, IFT74, LRRC19, TEK, EQTN, MOB3B, IFNK and LINGO2. In a further embodiment, the C9ORF72-mediated genes comprise PLAA, MOB3B and TEK.

TABLE 1
Representative list of genes located within about a
2 Mb window upstream or downstream of the location
of C9ORF72 (ENSG00000147894; chromosomal location of C9ORF72
9:27535640-27573866), was compiled from the GRCh38.p13
Homo sapiens Genome Assembly of EnsEmbl.

| Gene Symbol | Gene ID | Chromosomal Location |
|---|---|---|
| IZUMO3 | ENSG00000205442 | 9:24542952-24545946 |
| TUSC1 | ENSG00000198680 | 9:25676389-25678440 |
| CAAP1 | ENSG00000120159 | 9:26840685-26892803 |
| PLAA | ENSG00000137055 | 9:26903372-26947242 |
| IFT74 | ENSG00000096872 | 9:26947039-27062930 |
| LRRC19 | ENSG00000184434 | 9:26993136-27005672 |
| TEK | ENSG00000120156 | 9:27109141-27230174 |
| EQTN | ENSG00000120160 | 9:27284654-27297150 |
| MOB3B | ENSG00000120162 | 9:27325209-27529814 |
| IFNK | ENSG00000147896 | 9:27524314-27526498 |
| LINGO2 | ENSG00000174482 | 9:27948078-28670286 |

C9ORF72-mediated genes are located on human chromosome 9 within about 2 Mb upstream or downstream of the location of C9ORF72 (ENSG00000147894; chromosomal location of C9ORF72 9:27535640-27573866). Preferably, the C9ORF72-mediated gene is selected from the group consisting of IZUMO3, TUSC1, CAAP1, PLAA, IFT74, LRRC19, TEK, EQTN, MOB3B, IFNK and LINGO2 or a combination thereof. More preferably, the C9ORF72-mediated gene is selected from the group consisting of PLAA, MOB3B and TEK or a combination thereof.

Many genes are expressed in more than one subset of cells or tissues. The activity of transcription factors allows temporal and spatial regulation of these genes. Transcription factors bind to regulatory elements called enhancers, resulting in stimulation or enhancement, or alternatively repression of gene transcription. Enhancers contain the same regulatory elements that are found at the promoters of the genes that they regulate. Enhancers generally represent a modular arrangement of short sequence motifs, each interacting with a specific cellular transcription factor, which will be responsible for turning the transcription on or off in a different set of cells, or at different times in development. Enhancers can exert their functions in both orientations, regardless of their position from target genes. Proximal enhancers can be located a few tens of bp from their target promoter. In some instances, however, enhancers modulate DNA transcription over long distances, and can affect the transcription of genes located in cis as far as 2 Mb away on the same chromosome^{xiii}. The ability of enhancers to interact with promoters is not limited to genes located in cis on the same chromosome. Gene regulatory elements have been found to engage in direct physical interactions with target genes on other chromosomes^{iv}. Yet an important property of enhancers is that their readout is context-specific^{xv}, so too are the effects of mutations within them, that may alter a gene's expression in one tissue, but not in another. C9ORF72 hexanucleotide expansions can be directly anticipated to regulate C9ORF72-mediated genes (Table 1). The present invention provides a thorough examination of a database of transcription factor target genes and the results of a JASPAR analysis. The present invention thereby discloses that C9ORF72 hexanucleotide expansions affecting transcriptional mechanisms control the expression of C9ORF72-mediated genes.

The DNA structure of a sequence of five repeats of SEQ 1A, labelled as SEQ ID NO: 1, was analyzed for the presence of transcription factor binding sites using binding profiles from the JASPAR CORE database of experimentally defined transcription factor binding sites for eukaryotes (http://jaspar.genereg.net). A relative profile score threshold cut-off of 85% was used. A score was calculated for the probed sequence that provides a measure of similarity to the transcription factor consensus sequence. A transcription factor binding site was classified as “gained” or “de novo” if it is found by the JASPAR screen using the 85% threshold cut-off value. A summary of the results is shown in Table 2.

TABLE 2
JASPAR analysis of transcription factor binding sites in
SEQ ID NO: 1, representing five hexanucleotide repeats.
Model name refers to the name of the transcription factor.

| | | | | C90RF72-mediated |
|---|---|---|---|---|
| | | | | gene identified as |
| | Model | | Relative | transcription factor target |
| Model ID | name | Score | score | (CHEA, ENCODE) |
| MA0079.3 | SP1 | 8.846 | 0.892 | CAAP1, PLAA, TEK, IFT74 |
| MA0599.1 | KLF5 | 6.597 | 0.887 | |
| MA0146.2 | ZFX | 10.551 | 0.876 | PLAA |
| MA0039.2 | KLF4 | 8.221 | 0.876 | TUSC1 |
| MA0516.1 | SP2 | 11.234 | 0.876 | |
| MA0162.2 | EGR1 | 8.074 | 0.866 | CAAP1, IFT74, MOB3B, |
| | | | | TUSC1, PLAA |
| MA0741.1 | KLF16 | 8.783 | 0.861 | |
| MA0003.3 | TFAP2A | 6.587 | 0.858 | TUSC1 |

Table 2 shows the highest scoring transcription factor binding sites identified using the sequence of SEQ ID NO: 1. Transcription factor binding sites were identified for SP1, KLF5, ZFX, KLF4, SP2, EGR1, KLF16 and TFAP2A. Therefore, the C9ORF72 hexanucleotide expansions contain many more of the identified transcription factor binding sites compared to the C9ORF27 wild-type allele and thereby affecting the expression of C9ORF72-mediated genes.

The invention further discloses the presence of the transcription factor binding sites identified in Table 2 in the promoters of C9ORF72-mediated genes. To this end, the CHEA Transcription Factor Targets dataset»vi that includes low- and high-throughput transcription factor functional studies, was analyzed. Several C9ORF72-mediated genes bind transcription factors identified in the JASPAR analysis in their promoter regions. The transcription factor binding site for SP1 is present in the promoter of the C9ORF72-mediated genes CAAP1, PLAA, TEK and IFT74. The transcription factor binding site for ZFX is present in the promoter of the C9ORF72-mediated gene PLAA. The transcription factor binding site for KLF4 is present in the promoter of the C9ORF72-mediated gene TUSC1. The transcription factor binding site for EGR1 is present in the promoters of the C9ORF72-mediated gene CAAP1, IFT74, MOB3B, TUSC1 and PLAA. The transcription factor binding site for TFAP2A is present in the promoter of the C9ORF72-mediated gene TUSC1.

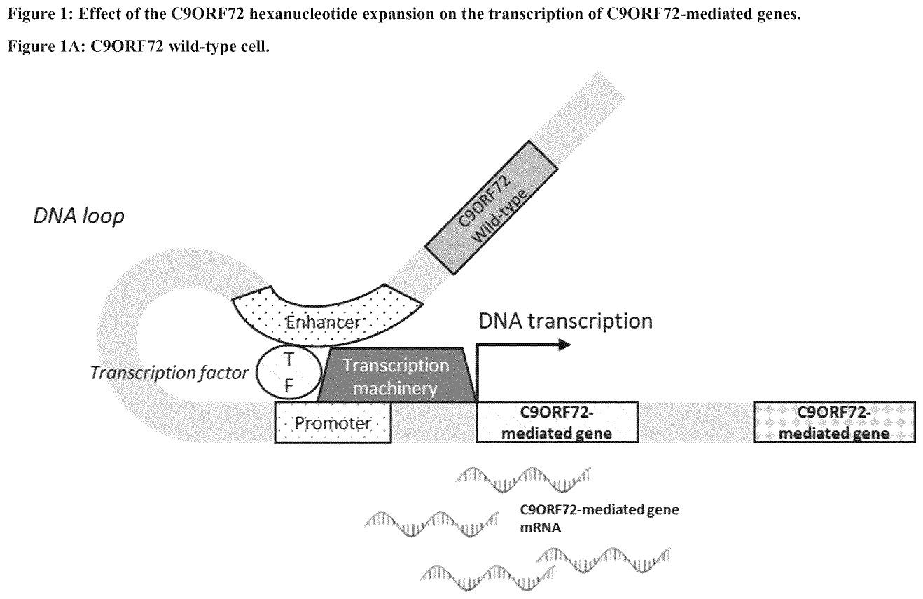

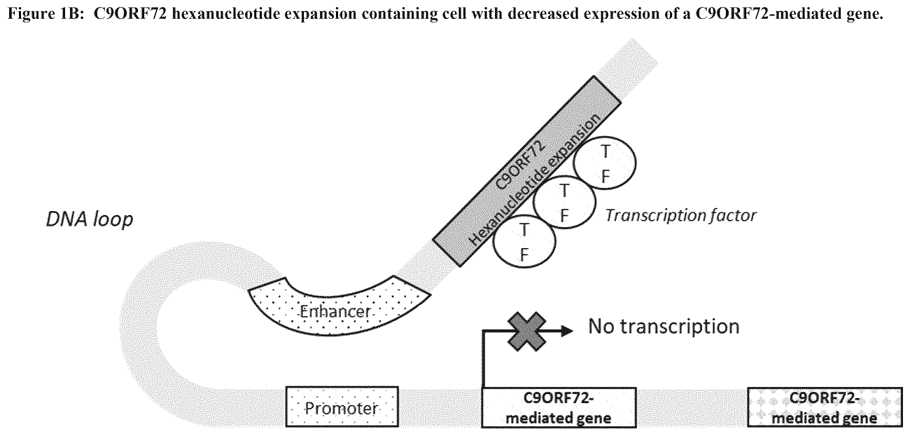

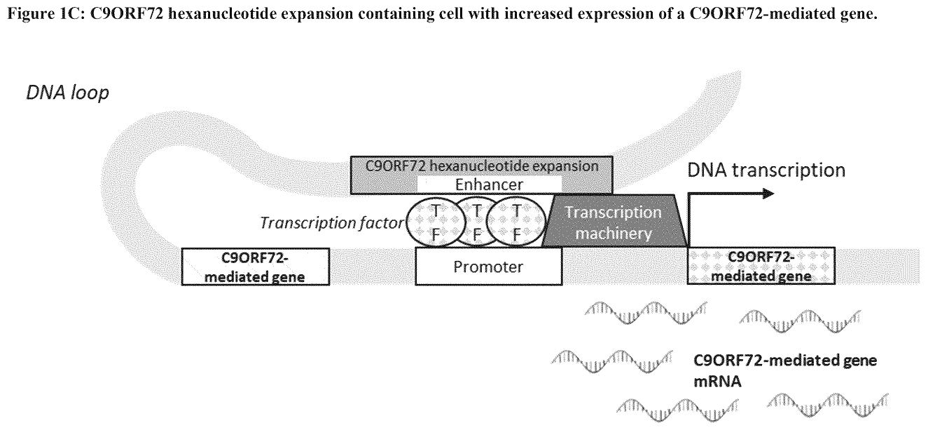

Transcriptional mechanisms affected by the C9ORF72 hexanucleotide expansion are present in proximal or distal enhancers and/or the promoter regulating the C9ORF72-mediated genes lying in the vicinity of the C9ORF72 hexanucleotide expansion (FIG. 1). Enhancers contain the same regulatory elements that are found at the promoters of the C9ORF72-mediated genes that they regulate (FIG. 1A). Presence of multiple transcription factor binding sites in the C9ORF72 hexanucleotide expansion decreases (FIG. 1B) or increases (FIG. 1C) enhancer activity and transcription of the C9ORF72-mediated genes.

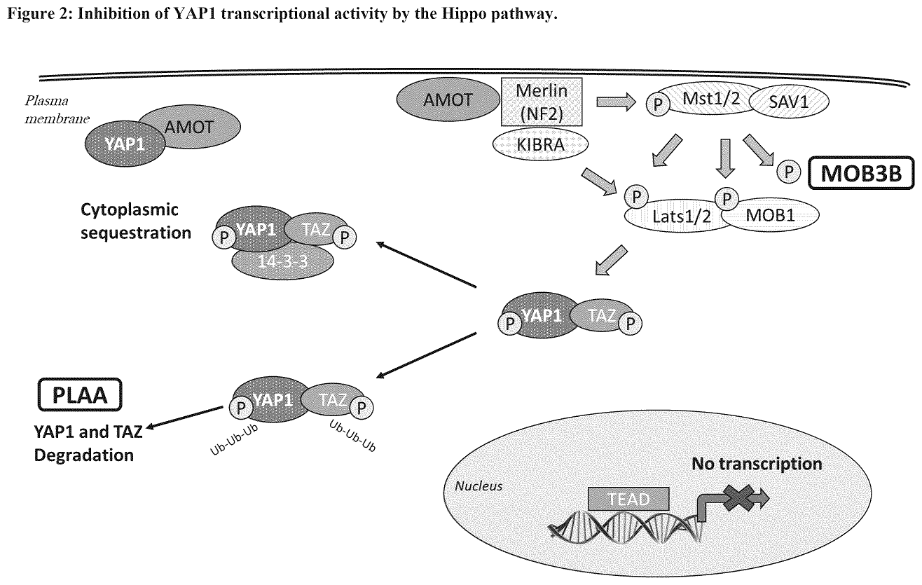

YAP1 (Yes Associated Protein 1; UniProtKB as Transcriptional coactivator YAP1, “YAP1_HUMAN” with the accession number P46937) is a transcriptional coactivator that regulates a variety of cellular processes, including cell spreading, and migration, glucose uptake and metabolism and mitochondrial biogenesis and remodeling. YAP1 is also involved in neuronal survival and controls the activation of pathways necessary for cytoskeletal rearrangements, dendritic spine expansion and synaptic plasticity. Transcriptional activity of YAP1 and its coactivator TAZ (WWTR1, WW Domain Containing Transcription Regulator 1) is regulated by the Hippo pathway (FIG. 2). YAP1 can be phosphorylated on the serine residue at position 109. This phosphorylated version of YAP1 is designated as phospho-Ser¹⁰⁹-YAP1. YAP1 can be phosphorylated on the serine residue at position 127. This phosphorylated version of YAP1 is designated as phospho-Ser¹²⁷-YAP1.

Core to the Hippo pathway is an evolutionarily conserved serine/threonine kinase signaling cascade. MST1/2 (Serine/Threonine Kinase 3 and 4, STK3/4) phosphorylate and activate LATS1/2 (Large Tumor Suppressor Kinase 1 and 2), which in turn phosphorylate and promote either ubiquitination and degradation or cytoplasmic retention of YAP1 and its transcription co-activator TAZ (WWTR1) via a 14-3-3 interaction. MST1/2 and LATS1/2 activation require the presence of coactivators, respectively regulatory SARAH domain containing Salvador homolog 1 (SAV1) and regulatory protein MOB1 (MOB kinase activator 1A and 1B). MOBIA and MOBIB play redundant biological roles. Merlin (neurofibromin 2/NF2) along with KIBRA (WWC1) can synergistically induce the phosphorylation of LATS1 and LATS2. AMOT (angiomotin), a protein from the motin family strongly interacts with YAP1 and sequesters it in the cytoplasm. Angiomotin can also bind to Merlin and release the auto-inhibition loop that blocks Merlin binding to LATS1/2 permitting subsequent LATS1/2 activation.

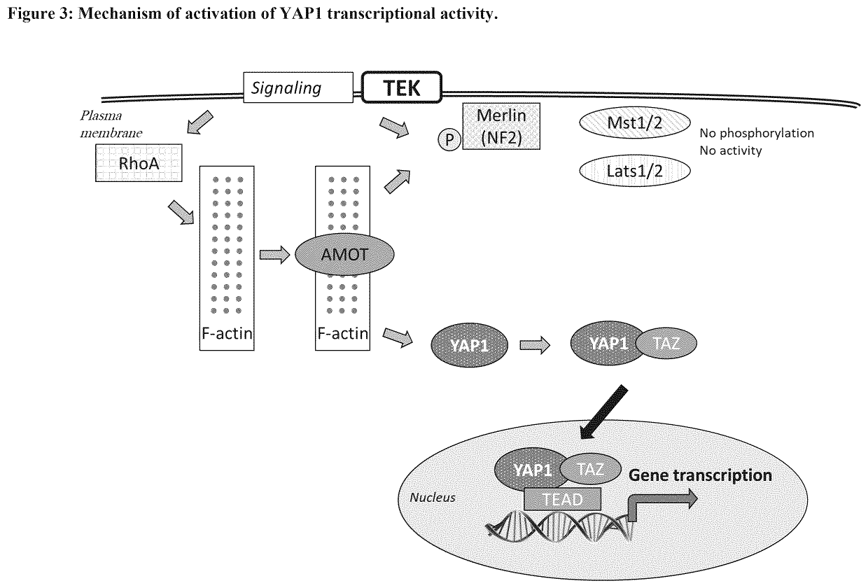

Unphosphorylated YAP/TAZ are able to translocate into the nucleus and interact with transcription factors from the TEAD family to induce targeted gene expression (FIG. 3). YAP1 nuclear translocation is an indirect effect of actin polymerization in response to RhoA activation. Two F-actin dependent events concomitantly allow YAP1/TAZ nuclear translocation^{xvii}. On one side, direct competition for binding with F-actin of AMOT allows the release of YAP1^{xviii}. On the other side, AMOT binding to F-actin impedes Merlin binding and activation. Integrins also inactivate Hippo signaling by inactivating Merlin through phosphorylation by ILK (integrin-linked kinase)^{xix}.

In the nucleus, YAP1 induces the expression of genes such as integrin subunits (αV, β1 and β3), and focal adhesion structural proteins (e.g., vinculin and zyxin)^{xx}, cell cycle markers such as cyclin A2, cyclin B1, cyclin D3 and cyclin kinase CDK1. YAP1 has also been shown to regulate a variety of shared target genes with MRTF-A/SRF (Myocardin Related Transcription Factor A/Serum Response Factor). Finally, YAP1 regulates the transcription of Rho GTPase Activating Protein 29 (ARHGAP29) that suppress the RhoA-LIMK-cofilin pathway^{xxi}, in a feedback mechanism^{xxii}. The present invention demonstrates by analysis of the CHEA Transcription Factor Targets dataset that genes linked to ALS are target genes of YAP1. These genes include C9ORF72, TUSC1, ALS2, ANXA11, CHMP2B, FUS and SOD1.

Many modulators of the YAP1/Hippo pathway exist. Some of the genes linked with familial ALS like ERBB4^{xxiii} and VCP associate with YAP1 and tune its transcriptional activity. The present invention discloses that the activities of at least three of the C9ORF72-mediated genes in Table 1 can surprisingly be linked to the YAP1/Hippo pathway, namely PLAA, MOB3B and TEK. Other genes in Table 1 could also interfere with the YAP1/Hippo pathway. Prior to the instant invention, none of these genes have been previously identified as potential targets for diagnosis or therapy of ALS, FTD, or both, in carriers of a C9ORF72 hexanucleotide expansion.

The present invention discloses that C9ORF72 hexanucleotide expansions alter transcription factor recognition motifs and act as enhancers causing improper transcription of one or several of C9ORF72-mediated genes in vulnerable neurons of carriers of a C9ORF72 nucleotide expansion with ALS, FTD, or both.

The present invention reveals that C9ORF72 hexanucleotide expansions alter the DNA properties and the effect of the distance between C9ORF72 and C9ORF72-mediated genes in its genomic vicinity. This altered distance affects transcription factor binding sites and thereby affects the expression of C9ORF72-mediated genes (FIG. 1). Altered transcription factor binding sites can act in combination with other gene transcription mechanisms including but not limited to, chromatin looping, histone modifications, and regulation of the transcription machinery. These altered transcription mechanisms act as enhancers and insulators causing improper transcription of one or several of C9ORF72-mediated genes in ALS, FTD, or both, vulnerable neurons containing a C9ORF72 hexanucleotide expansion.

The present invention is based on the discovery of detailed knowledge of the underlying genetic mechanism for development of ALS, FTD, or both, in carriers of a C9ORF72 hexanucleotide expansion, as discussed above. In particular, the present invention has provided methods for more accurate diagnosis and/or treatment for ALS, FTD, or both, in carriers of a C9ORF72 hexanucleotide expansion. This discovery also provides methods for preventing development of ALS, FTD, or both, in individuals that are carriers of a C9ORF72 hexanucleotide expansion.

Based on the analysis of the gene structure, the present invention demonstrates that C9ORF72 hexanucleotide expansions interfere with transcriptional mechanisms controlling the expression of C9ORF72-mediated genes. Based at least in part by this discovery, one aspect of the invention provides a method of modulating the transcriptional mechanisms to inter alia stabilize or improve the motor function, cognitive function and other disease manifestations of carriers of a C9ORF72 hexanucleotide expansion with ALS, FTD, or both.

In one embodiment, modulation of C9ORF72-mediated gene expression is achieved by contacting a cell that is expressing a C9ORF72-mediated gene with a molecule that is capable of interfering with transcriptional mechanisms emanating from C9ORF72 hexanucleotide expansions. In some embodiments, the cell is a neuronal cell, a neuronal progenitor cell, a differentiated neuron, an oligodendrocyte, a fibroblast, a lymphocyte, a human embryonic kidney cell or another cell commonly used in drug discovery and development efforts known to one skilled in the art.

Still in another embodiment the invention provides a method for modulating the expression of a C9ORF72-mediated gene. It should be appreciated that the term “modulating” when referring to a gene expression means the amount of gene transcribed and/or translated is modulated (i.e. increased or decreased) by at least about 10%, typically by at least about 20%, or substantially completely (i.e., >90% reduction or 10-fold increase) in the presence of a molecule of the invention compared to the same cell line in the absence of the molecule. One skilled in the art can readily measure the degree of modulation using any of the methods that are known.

Still in another embodiment the invention provides a method for modulating the activity of a C9ORF72-mediated gene. It should be appreciated that the term “modulating” when referring to measuring the activity of a C9ORF72-mediated gene means the level of activity is modulated (i.e., increased or decreased) by at least about 10%, typically by at least about 20%, or substantially completely (i.e., >90% reduction or 10-fold increase) in the presence of a molecule of the invention compared to the same cell line in the absence of the molecule. One skilled in the art can readily measure the degree of modulation using any of the methods that are known.

The molecule used for modulating the expression or the activity of a C9ORF72-mediated gene includes a molecule known to one skilled in the art, such as, but not limited to, small molecules, oligonucleotides (including short interfering RNAs, RNAs, long non-coding RNAs and aptamers), peptides, polypeptides (including aptamers, zinc fingers and fragments thereof), proteins (including antibodies and fragments thereof) as well as derivatives or modified forms thereof. In some embodiments, a molecule is delivered using a viral vector. In one instance, the oligonucleotide is a single-strand oligonucleotide. Yet in another instance, the decoy oligonucleotide is a double-strand oligonucleotide.

In one embodiment, the molecule is a small molecule. A small molecule is an organic compound with a molecular weight of less than 2000 Dalton, preferably less than 1500 Dalton and most preferably 900 Dalton or less.

In another embodiment, the molecule is an oligonucleotide. The term “oligonucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally-occurring nucleotides. Examples of such analogs include, without limitation, phosphorothioates, phosphoramidates, methyl phosphonates, chiral-methyl phosphonates, 2-O-methyl ribonucleotides, a locked-nucleic acid-modified oligonucleotide, and peptide-nucleic acids (PNAs). As used herein, the term “locked nucleic acid” refers to a nucleic acid in which the ribose moiety is modified with an extra bridge connecting the 2′-oxygen and 4′-carbon.

In another embodiment, the oligonucleotide is fused to a cell penetrating peptide or is a peptide nucleic acid oligonucleotide, a locked-nucleic acid-modified oligonucleotide, and combinations thereof.

In one embodiment, an oligonucleotide is a ribonucleotide polymer also known as ribonucleic acid (RNA). In some instances, the RNA is a short interfering RNA (siRNA), a messenger RNA (mRNA), a microRNA (miRNA) or a long non-coding RNA (lncRNA).

In a further embodiment, the molecule is a peptide, a polypeptide or a protein. A peptide is a compound in which amino acids, including natural, artificial and modified amino acids, are linked by a peptide bond. In some embodiments, the peptide contains two to twenty linked amino acids. In another embodiment the peptide is called a polypeptide and contains up to fifty linked amino acids. In a further embodiment, the peptide contains more than fifty linked amino acids and is called a protein. In some instances, the protein is a C9ORF72-mediated gene expression product or a derivative thereof. In some instances, the protein is an antibody or a fragment thereof. In some instances, the peptide, the polypeptide or the protein binds to the C9ORF72 hexanucleotide extension, more preferably to a sequence of SEQ ID NO:1, most preferably to a segment of 9 to 30 nucleotides within the SEQ ID NO:1. In some instances, the peptide, the polypeptide or the protein is a zinc finger protein or a fragment thereof.

The molecule can be provided in a pharmaceutically acceptable carrier. The terms “pharmaceutically acceptable carrier” and “pharmaceutically acceptable excipient” are used interchangeably herein and refer to an excipient that is useful in preparing a pharmaceutical composition that is generally safe, non-toxic and neither biologically nor otherwise undesirable, and includes excipient that is acceptable for human pharmaceutical use.

In some embodiments, the molecule is delivered by a viral vector. In some embodiments, the viral vector is based on an adeno-associated virus, a retrovirus, a lentivirus, an adenovirus or a herpes simplex virus. In some embodiments, the viral vector encodes a protein including but not limited to an expression product of a C9ORF72-mediated gene or a fragment thereof, an antibody or a fragment thereof, or a zinc finger protein or a fragment thereof. In another embodiment, the viral vector encodes an RNA, including but not limited to a siRNA, a mRNA, a miRNA or a lncRNA.

Still in other embodiments, the cell comprises a neuronal cell, a neuronal progenitor cell, a differentiated neuron, an oligodendrocyte, a fibroblast, a lymphocyte, a human embryonic kidney cell or another cell commonly used in drug discovery and development efforts known to one skilled in the art. Methods of the invention are applicable to a neuronal cell, a neuronal progenitor cell, a differentiated neuron, an oligodendrocyte, a fibroblast, a lymphocyte, a human embryonic kidney cell or another cell commonly used in drug discovery and development efforts known to one skilled in the art.

The invention provides methods to identify a lead candidate for drug development for treatment of ALS, FTD, or both in carriers of a C9ORF72 hexanucleotide expansion. The method can involve an in vitro or a biochemical assay that does not contain whole cells. The assay may contain cell extracts or other cellular components. The assay may also comprise a substantially purified gene or gene expression product. In this context “substantially purified” refers to a composition where the gene or gene expression product is present at least 10-fold higher than in any naturally occurring context, and wherein elements of the natural context have been removed, yet wherein the gene or gene expression product retains the ability to participate in a biochemical reaction which it normally engages in its natural context.

One skilled in the art having read the present disclosure can readily recognize suitable in vitro assay conditions necessary to identify a molecule that can modulate binding of a transcription factor to a C9ORF72 hexanucleotide expansion. Typically, it will be an in vitro binding assay in which direct binding of a transcription factor to an immobilized oligonucleotide representing the C9ORF72 hexanucleotide expansion is measured. Exemplary suitable in vitro assays include, but are not limited to, a surface plasmon resonance and an electrophoretic mobility shift assay (EMSA). Briefly, in EMSA a radioactively labeled C9ORF72 hexanucleotide expansion oligonucleotide is incubated with a recombinant transcription factor and a molecule to be tested as being a possible lead drug candidate. The ability of the molecule to modulate binding of the recombinant transcription factor to the labeled C9ORF72 hexanucleotide expansion oligonucleotide is then analyzed by gel electrophoresis. If the transcription factor is bound to the labeled C9ORF72 hexanucleotide expansion oligonucleotide (that is the molecule did not significantly modulate binding), the observed mobility of the labeled C9ORF72 hexanucleotide expansion oligonucleotide is shifted towards higher molecular weight.

The invention provides methods to identify a lead candidate for drug development for treatment of ALS, FTD, or both in carriers of a C9ORF72 hexanucleotide expansion involving whole cells. In some instances, the whole cells carry C9ORF72 hexanucleotide expansions. The assay may contain cell extracts or other cellular components. The assay may also comprise a recombinant C9ORF72-mediated gene expression product wherein the gene or gene expression product retains the ability to participate in a biochemical or cellular reaction which it normally engages in its natural context.

High-Throughput Screening Assays: Screening assays of the invention are designed to identify modulation of a function, activity or amount of an C9ORF72-mediated gene or gene expression product, e.g., the mRNA or the protein generated from the gene sequence. As used herein the term “modulation” means any change in activity of a function or amount of the transcribed gene, mRNA or protein, (together which are sometimes called the “target”) including any change in transcription rate or expression level, and includes inhibition or activation, and antagonist and agonist effects on the biochemical or biological activity of the target. Throughout this disclosure, the term “change” when referring to any biological activity, e.g., C9ORF72-mediated gene expression or activity of C9ORF72-mediated gene expression product, means the value is statistically different from a control (i.e., p<0.25, often p<0.1, and more often p<0.05). The term “control” of gene expression or activity of a gene expression product refers to a standard level against which gene expression or the activity of the gene expression product, respectively, in cell is or can be compared. In some embodiments, the control can be cells carrying the C9ORF72 wild-type allele, meaning the C9ORF72-mediated gene expression level and/or activity measured in cells carrying the C9ORF72 hexanucleotide expansions is compared to the expression level and/or activity in C9ORF72 wild-type cells. This allows a determination based on the expression or biological activity against cells or subject with the C9ORF72 wild-type.

This specification discloses diverse functions of C9ORF72-mediated genes, either known from the art or implied from proteins of the same class, that may be used to assess modulation. Some functions may be assessed directly, such as the catalyzing of a specific reaction, or less directly, such as by measuring the accumulation of a downstream product. Many assay designs are available to those skilled in the art. Preferred assays are optimized for speed, efficiency, signal detection and low reagent consumption. (Zhang et al. (1999) J. Biomolec. Screen. 4 (2): 67). Assays can be reporter assays measuring gene transcription, gene translation or a biological activity of the gene expression product. Assays can be developed in a neuronal cell, a neuronal progenitor cell, a differentiated neuron, an oligodendrocyte, a fibroblast, a lymphocyte, a human embryonic kidney cell or another cell commonly used in drug discovery and development efforts known to one skilled in the art. The examples below include description of assays for accumulation of phosphorylated YAP1 protein with phosphorylation at the serine residue at position 127 (phospho-Ser127-YAP1), or the serine residue at position 109 (phospho-Ser109-YAP1), or both, or in combination with phosphorylation at other residues. Phosphorylated YAP1 protein is an example of one such downstream product which can be measured in cells to identify molecules which modulate the expression or activity of one or more of the C9ORF72-mediated gene products. Based on the invention herein, those of skill in the art can now predictably develop screening assays for potential ALS, FTD, or both, therapeutic agents in carriers of C9ORF72 hexanucleotide expansions based on assays designed to measure if the compound modulates the protein expression or functions disclosed herein, and other functions known or to be discovered now that their significance is understood.

In some embodiments, the screening assays of the invention, either cellular, cell extract or biochemically based with substantially purified genes or gene expression products, are designed for testing a plurality of compounds (e.g., millions) through high-throughput screening of chemical libraries.

Chemical libraries of test compounds that may be screened to identify a modulator can be obtained from numerous available resources or using any of the numerous approaches in library synthesis methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145). See also Dolle et al. (2010) Comprehensive Survey of Chemical Libraries for Drug Discovery and Chemical Biology: 2009. J. Comb. Chem., 2010, 12 (6), pp 765-806.

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Test compounds which successfully modulate the activity or expression level of a C9ORF72-mediated gene or expression product are attractive candidates for further investigation and secondary screening in alternative assays for potential use in treating ALS, FTD, or both, in carriers of C9ORF72 hexanucleotide expansions. Compounds are considered “potentially useful for treatment” when first identified in a screening assay, because it is well known that initial successful hits rarely contain all the required features for a successful pharmaceutical. They are however extremely useful to allow researchers to identify a chemical core structure shared among compounds that effectively modulates the target activity. Typically, when a core structure is identified, an extensive library of possibly thousands of related compounds is further developed with the aim of identifying a lead compound that meets all the criteria for a successful pharmaceutical candidate. The assay is used repeatedly through many rounds of screening of up to millions of compounds to ultimately identify a small group of lead compounds, one of which may eventually become an approved therapeutic agent.

In some embodiments of the invention, a possible lead molecule for treatment of ALS, FTD, or both, in carriers of a C9ORF72 hexanucleotide expansion identified by methods of the invention has 50% inhibition or activation concentration (IC₅₀ or EC₅₀) of about 500 μM or less, typically about 100 μM or less, often about 50 μM or less, more often about 10 μM or less, and most often about 500 nM or less.

Still another aspect of the invention provides a method for modulating C9ORF72-mediated expression of a gene in a cell. It should be appreciated that throughout this disclosure the term “C9ORF72-mediated expression of a gene” refers to expression of a gene caused by, due to, or mediated by a C9ORF72 hexanucleotide expansion. Expression of a gene refers to induction or repression of said gene. Such a method includes contacting a cell that is expressing or is capable of expression a gene mediated by C9ORF72 hexanucleotide expansion with a molecule that is capable of modulating (i.e., increasing or decreasing) the expression of said gene. Modulation of expression of said gene means an increase or a decrease of the level of the expression product of said gene. In one embodiment, the gene that is expressed by the C9ORF72 hexanucleotide expansion is located on human chromosome 9 within about a 2 Mb window upstream or downstream of the location of C9ORF72 (ENSG00000147894; chromosomal location of C9ORF72 9:27535640-27573866). Exemplary C9ORF72-mediated genes include, but are not limited to IZUMO3, TUSC1, CAAP1, PLAA, IFT74, LRRC19, TEK, EQTN, MOB3B, IFNK and LINGO2. In particular, methods of the invention include modulating expression of the following genes that are mediated by C9ORF72 hexanucleotide expansions: IZUMO3, TUSC1, CAAP1, PLAA, IFT74, LRRC19, TEK, EQTN, MOB3B, IFNK and LINGO2. In a further embodiment, the C9ORF72-mediated gene comprises PLAA, MOB3B, TEK, EQTN, IZUMO3 or a combination thereof.

Clinical use of methods of the invention includes a method for treating a subject carrying a C9ORF72 hexanucleotide expansion and suffering from ALS, FTD, or both. Methods of the invention include determining the C9ORF72 genotype present in said subject; and (a) if said subject carries an allele of a C9ORF72 hexanucleotide expansion, administering the subject with a molecule that is capable of modulating the expression or activity of a C9ORF72-mediated gene; or (b) if the subject is not a carrier of a C9ORF72 hexanucleotide expansion, administering the subject with a molecule that is different from the molecule that modulating the expression or activity of a C9ORF72-mediated gene. In some embodiments, the step of determining the C9ORF72 genotype in the subject comprises a step of determining whether the subject is homozygous or heterozygous for the C9ORF72 hexanucleotide expansion.

The molecule of the invention can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the molecule of the invention may be incorporated with excipient and used in the form of ingestible tablets, buccal tablets, troches, capsules, elixirs, suspensions, syrups, wafers, and the like. Such compositions and preparation can contain at least 0.1% of molecule of the invention. The percentage of the compositions and preparation can, of course, be varied and can conveniently be between about 1 to about 10% of the weight of the unit. The amount of molecule of the invention in such therapeutically useful compositions is such that a suitable dosage will be obtained. Preferred compositions or preparations according to the present invention are prepared such that an oral dosage unit form contains from about 1 to about 1000 mg of molecule of the invention.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the molecule of the invention, sucrose as a sweetening agent, methyl and propylparabens as preservatives, a dye and flavoring such as cherry or orange flavor. Any material used in preparing any dosage unit form should be pharmaceutically pure and substantially non-toxic in the amounts employed. In addition, the molecule of the invention can be incorporated into sustained-release preparations and formulation.

The molecule of the invention can also be administered parenterally. Solutions of the molecule of the invention as a free base or pharmacologically acceptable salt can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringeability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating the molecule of the invention in the required amount in the appropriate solvent with various other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

The physician will determine the dosage of the molecule of the invention which will be most suitable for prophylaxis or treatment and it will vary with the form of administration and the particular molecule chosen, and also, it will vary with the particular patient under treatment. The physician will generally wish to initiate treatment with small dosages by small increments until the optimum effect under the circumstances is reached. The therapeutic dosage can generally be from about 0.1 to about 1000 mg/day, and preferably from about 10 to about 100 mg/day, or from about 0.1 to about 50 mg/Kg of body weight per day and preferably from about 0.1 to about 20 mg/Kg of body weight per day and can be administered in several different dosage units. Higher dosages, on the order of about 2× to about 4×, may be required for oral administration.

The molecule of the invention can also be administered directly to the brain using stereotactic surgery.

The molecule of the invention can also be delivered by a viral vector.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Transcription factor recognition motifs in a (9ORF72 hexanucleotide expansion: The C9ORF72 hexanucleotide expansions were analyzed for presence or absence of transcription factor binding sites by using the JASPAR database of human transcription factor recognition motifs. This study allowed the determination of whether new transcription factor binding sites are created by the C9ORF72 hexanucleotide expansions. The DNA sequence contained in SEQ ID:NO: 1 for transcription factor binding sites was analyzed using binding profiles from the JASPAR CORE database of experimentally defined transcription factor binding sites for eukaryotes (http://jaspar.genereg.net/) using a relative profile score threshold cut-off of 85%. A transcription factor binding site was classified as “gained” or “de novo” in a C9ORF72 hexanucleotide expansions if it was found by the JASPAR screen using the 85% threshold cut-off value. A summary of the results is shown in Table 2.

The C9ORF72 hexanucleotide expansion creates several de novo binding sites. Among them are recognition sites for the transcription factors SP1, KLF5, ZFX, KLF4, SP2, EGR1, KLF16 and TFAP2A.

These examples demonstrate that C9ORF72 hexanucleotide expansions have profound effects on the presence or absence of transcription factor recognition motifs.

As can be clearly seen, the C9ORF72 hexanucleotide expansions create de novo binding sites for transcription factors. By binding to the genomic location of C9ORF72 hexanucleotide expansions and the proximal promoter region of one of the genes harboring transcription factor binding sites this transcription factor leads to inappropriate (increase or decrease) expression of C9ORF72-mediated genes (FIG. 1). Thus, some aspects of the invention provide a method of treating ALS, FTD, or both, in carriers of a C9ORF72 nucleotide expansion by decreasing or increasing the expression of one or more C9ORF72-mediated genes.

A list of the genes located within about a 2 Mb window upstream or downstream of the location of C9ORF72 (ENSG00000147894; chromosomal location of C9ORF72 9: 9:27535640-27573866) was compiled from the GRCh38.p13 Homo sapiens Genome Assembly of EnsEmbl and is provided in Table 1.

For each of the genes within 2 Mb of C9orf72, the CHEA (ChIP Enrichment Analysis) Transcription Factor Targets dataset and ENCODE Transcription Factor Target dataset provided by the Harmonizome (http://amp.pharm.mssm.edu/Harmonizome/about), a publicly available collection of information and research findings about genes and proteins from 114 datasets collected from 66 online resources (e.g. genomics, transcriptomics, proteomics, metabolomics, from cells, tissues, model organisms, and patients) was examined. CHEA integrate the results of experiments such as ChIP-chip, ChIP-PET and DamID which profile the binding of transcription factors to DNA at a genome-wide scale and provide a list of the transcription factors binding the promoter of the gene of interestMiv. The ENCODE Transcription Factor Targets compile transcription factor DNA-binding sites identified by ChIP-seq. These analyses revealed that ZFX binds to the promoter of PLAA, that KLF4 and TFAP2A bind to the promoter of TUSC1, that SP1 binds to the promoters of CAAP1, PLAA, TEK, IFT74 and that EGR1 binds to the promoters of TUSC1, PLAA, MOB3B, CAAP1 and IFT74 (Table 2).

The presence of transcription factor binding sites in C9ORF72 hexanucleotide expansions affect C9ORF72-mediated gene transcription by competitive binding of transcription factors to this expanded region, to the detriment of binding to promoters and enhancers in C9ORF72-mediated genes. Also, the hexanucleotide repeat expansion in C9orf72 gene can adopt atypical secondary structures^{XXV} susceptible to impair transcription factors binding, enhancer-promoter interactions and ultimately expression of the genes within the loop^{xxvi}. Presence of the expansion is likely to affect regional CpG methylation (hypo, alternatively hypermethylation), heterochromatin boundaries and gene expression^{xxvii}. Enhancers themselves are abundantly transcribed into regulatory eRNAs that can interact with selective transcription factors and chromatin regulators to promote gene activation^{xxviii}. Moreover, presence of the expansion leads to clustering of transcription factor binding sites at the enhancer. Such clusters are known to serve as regulatory hubs^{xxix}. In some instances, introduction of supplementary transcription factor binding sites in the expansion causes reduces gene expression^{xxx}.

C9ORF72-mediated genes function in the YAP1 Hippo pathway: A list of the genes located within about a 2 Mb window upstream or downstream of the location of C9ORF72 (ENSG00000147894; chromosomal location of C9ORF72 9:27535640-27573866), was compiled from the GRCh38.p13 Homo sapiens Genome Assembly of EnsEmbl and is provided in Table 1. The present invention provides an analysis of these genes and discloses that unexpectedly three of these genes are directly linked to the YAP1/Hippo activation pathway, namely PLAA, MOB3B and TEK. Other genes in Table 1 could also interfere with the YAP1/Hippo activation pathway.

YAP1/Hippo specific interference (PLAA): The PLAA gene is located on chromosome 9, upstream of C9ORF72. The expression of PLAA in neurons carrying a C9ORF72 hexanucleotide expansion is regulated by the transcriptional enhancer activity of C9ORF72 hexanucleotide expansion. The PLAA gene lies 0.63 Mb upstream from C9ORF72 on chromosome 9. PLAA (Phospholipase A2 Activating Protein UniProtKB “PLAP HUMAN” with the accession number Q9Y263). PLAA forms a functional complex with VCP, another gene involved in ALS, and plays a role in the sorting and degradation of ubiquitinated proteins, maturation of ubiquitin-containing autophagosomes and clearance of ubiquitinated protein by autophagy^{xxxi} is also associated with HDAC6, and involved in the formation of stress granules^{xxxii} involved in the formation of protein aggregates in ALS. Dysregulated expression of PLAA affects YAP1 degradation either through the proteasome or by autophagy, ultimately resulting in changes in the transcription of YAP1 target genes. Hence, the C9ORF72-mediated gene PLAA is clearly involved in a pathway affected in ALS. The present invention unexpectedly places a C9ORF72-mediated gene, PLAA, in the YAP1 functional pathway in C9ORF72 hexanucleotide expansion carriers.

These and other known biological activities of PLAA can be employed by those skilled in the art to design screening assays to screen for test compounds that modulate these activities, thereby identifying potential therapeutic agents for the treatment of carriers of a C9ORF72 hexanucleotide expansion for ALS, FTD, or both. Standard reporter assays incorporating the gene or gene expression product can also be employed.

YAP1 Hippo specific interference (MOB3B): The MOB3B gene is located on chromosome 9, upstream of C9ORF72. The expression of MOB3B in neurons carrying a C9ORF72 hexanucleotide expansion is regulated by the transcriptional enhancer activity of the C9ORF72 hexanucleotide expansion. The MOB3B gene lies 0.21 Mb upstream from C9ORF72 on chromosome 9. MOB3B (MOB Kinase Activator 3B; UniProtKB “MOB3B_HUMAN” with the accession number Q86TA1) is a member of the human MOB protein family which consists of six members: MOB1A/1B, MOB2, and MOB3A/3B/3C. MOB3B is thought to be a tumor suppressor. It can be phosphorylated by MST1/2^{xxxiii}, and binds^{xxxiv} Niv and modulates LATS1 expression^{xxxv}. Hence, unexpectedly, a C9ORF72-mediated gene, MOB3B, is a modulator of LATS1/2 activity, and consequently YAP1 phosphorylation, YAP1 nuclear translocation and YAP1 activity.

These and other known biological activities of MOB3B can be employed by those skilled in the art to design screening assays to screen for test compounds that modulate these activities, thereby identifying potential therapeutic agents for the treatment of carriers of a C9ORF72 hexanucleotide expansion for ALS, FTD, or both. Standard reporter assays incorporating the gene or gene expression product can also be employed.

YAP1 Hippo specific interference (TEK): The TEK gene is located on chromosome 9, upstream of C9ORF72. The expression of TEK in neurons carrying a C9ORF72 hexanucleotide expansion is regulated by the transcriptional enhancer activity of C9ORF72 hexanucleotide expansion. The TEK gene lies 0.43 Mb upstream from C9ORF72 on chromosome 9. TEK (TEK Receptor Tyrosine Kinase; UniProtKB “TIE2_HUMAN” with the accession number Q02763) is a cell surface receptor of angiopoietin and controls the formation of focal adhesion complexes, and activation of PTK2/FAK and of PI3K-AKT pathways. TEK is predominantly expressed in endothelial cells where it regulates angiogenesis and vascular remodeling. However, non-vascular functions of angiopoietin-TEK signaling have recently been uncovered^{xxxvi} Angiopoietins acting through their receptor TEK can directly support cell adhesion mediated by integrins. Hence, unexpectedly the dysregulated expression of a C9ORF72-mediated gene, the angiopoietin receptor TEK, affects integrin signaling, focal adhesion kinase signaling^{xxxvii} and merlin phosphorylation. Merlin phosphorylation in turn affects LATS1/2 activation, YAP1 phosphorylation, nuclear translocation and transcriptional activity.

These and other known biological activities of TEK can be employed by those skilled in the art to design screening assays to screen for test compounds that modulate these activities, thereby identifying potential therapeutic agents for the treatment of carriers of a C9ORF72 hexanucleotide expansion for ALS, FTD, or both. Standard reporter assays incorporating the gene or gene expression product can also be employed.

These examples demonstrate that C9ORF72 hexanucleotide expansions have profound effects on the presence or absence of transcriptional mechanisms leading to inappropriate (increase or decrease) expression of C9ORF72-mediated genes. Thus, some aspects of the invention provide a method of treating carriers of C9ORF72 hexanucleotide expansions for ALS, FTD, or both, by decreasing or increasing the expression of one or more C9ORF72-mediated genes.

Determination of expression levels of expression products encoded by (9ORF72-mediated genes: Human induced pluripotent stem cells (iPSC) with a known genotype of C9ORF27 are cultivated and differentiated into motor neurons. These cells can be derived from a subject carrying a C9ORF72 hexanucleotide expansion with ALS, FTD, or both, or from a healthy subject carrying a C9ORF72 hexanucleotide expansion. These cells can also be derived from cells carrying the C9ORF72 wild-type allele by introducing the C9ORF72 hexanucleotide expansion using molecular biology methods know to one skilled in the art including but not limited to CRISPR/CAS9 technology. There are numerous protocols available to the one skilled in the art to perform cultivation of iPSC and achieve differentiation into neurons, for example to motor neurons. Cells are typically received in cryovials, cells are then thawed and seeded in an appropriate culture vessel at a density of approximately 50,000 cells/cm². Cells are then grown in the presence of a neuronal differentiation medium for several days, typically three days, and cultivated for a further several weeks in the presence of a neuronal maintenance medium. Cells are assessed several weeks after differentiation for neuronal and synaptic marker expression, typically after 35 days. Neuronal markers are typically Tuj1 and MAP2, while synaptic markers are PSD-95 (postsynaptic terminals) and synaptophysin (presynaptic terminals). Commonly used markers to demonstrate differentiation to motor neurons are HB9 (early marker) and ChAT (choline acetyl transferase, mature marker). Cell cultures containing a C9ORF72 hexanucleotide expansion or the C9ORF72 wild-type allele are then analyzed for the expression of C9ORF72-mediated genes. From all the neuronal cultures the media are removed and stored under appropriate conditions known to the skilled artisan. The neurons are then rinsed with an appropriate buffer, for example phosphate-buffered saline, and lysed. The lysate is then analyzed for presence of the expressed protein encoded by the C9ORF72-mediated gene using one of the numerous methods available to the one skilled in the art. Cell lysates are prepared and mixed with an appropriate loading buffer. The levels of specific proteins in the lysate are determined by a combination of sodium dodecyl sulfate polyacrylamide gel electrophoresis (SDS-PAGE) of the lysates with a specific detection method (Western blot) using antibodies directed to the proteins to be analyzed. The levels of PLAA, MOB3B or TEK or any of the genes listed in Table 1 in neurons with a C9ORF72 hexanucleotide expansion are compared to levels in neurons with the C9ORF72 wild-type allele. The expression levels of the C9ORF72-mediated gene are also determined by quantification of messenger RNA (mRNA) levels using quantitative polymerase chain reaction (qPCR) and compared in neuronal cells carrying the C9ORF72 hexanucleotide expansion versus the C9ORF72 wild-type allele. Expression levels of some of the C9ORF72-mediated genes can also be measured in the media collected from neuronal cell cultures and compared between media from C9ORF72 hexanucleotide expansion bearing neurons and C9ORF72 wild-type allele bearing neurons.

In order to assess the activity of a molecule or a pharmaceutical composition on the expression of a C9ORF72-mediated gene the same cell culture conditions are employed. However, in this case the cell culture is incubated for several days, typically two days, most typically for twelve to 24 hours with a molecule before medium collection and lysate processing. In some instances, shorter incubation times lasting hours or minutes can be employed. The pharmaceutical composition can contain a molecule selected from, but not limited to, small molecules, oligonucleotides (including short interfering RNAs, RNAs and aptamers), peptides, polypeptides (including aptamers, zinc fingers and fragments thereof), proteins (including antibodies and fragments thereof) as well as derivatives or modified forms thereof.

It is readily apparent to one skilled in the art that this described method is not limited to neuronal cultures but can be applied to a neuronal cell, a neuronal progenitor cell, a differentiated neuron, an oligodendrocyte, a fibroblast, a lymphocyte, or any other cells collected from a patient with ALS, FTD, or both, who is a carrier of a C9ORF72 hexanucleotide expansion, or cells derived from cells collected from a human subject such as induced pluripotent stem cells derived from fibroblasts or plasma cells. It is readily apparent that this assay can be performed using blood, plasma, serum, cerebral spinal fluid or isolated blood cells collected from human subjects carrying a C9ORF72 hexanucleotide expansion, and can be used to diagnose ALS, FTD, or both, in these subjects. It is further apparent to one skilled in the art that this assay can be performed with human embryonic kidney cells, fibroblasts or other cells commonly used in drug discovery and development.

Determination of the activity of C9ORF72-mediated genes in a cellular assay: Differentiated human neurons or differentiated human neurons derived from human induced pluripotent stem cells, neuronal progenitor cells, cells of a cell line engineered to carry a C9ORF72 hexanucleotide expansion are maintained in culture. Alternatively, non-human mammalian cells, including but not limited to the species mouse, rat or non-human primates, of a cell line engineered to carry a C9ORF72 hexanucleotide expansion are maintained in culture. Specifically, human motor neurons with a C9ORF72 hexanucleotide expansion and motor neurons with a C9ORF72 wild-type allele are cultured.

Levels of total YAP1, or phospho-Ser127-YAP1, or phospho-Ser109-YAP1 are determined in the lysate of cultured cells by a combination of sodium dodecyl sulfate polyacrylamide gel electrophoresis (SDS-PAGE) of the lysates with a specific detection method using antibodies directed to total YAP1 or phospho-Ser127-YAP1 or phospho-Ser109-YAP1, using Western blots. The one skilled in the art knows of a multitude of alternative methods to determine protein levels in cell lysates.

The activity of a molecule on modulation of the C9ORF72-mediated gene's activity or expression level is measured by the levels of total YAP1, or phospho-Ser127-YAP1, or phospho-Ser109-YAP1 in cells with a C9ORF72 hexanucleotide expansion that were treated with vehicle containing said molecule and compared to the levels in control cells treated with vehicle not containing said molecule. The control cells can carry the C9ORF72 wild-type allele or a C9ORF72 hexanucleotide expansion. Typically, the cell culture is incubated for 1 to 48 hours with the molecule. The molecule used for modulating the expression or activity of a C9ORF72-mediated gene includes, but is not limited to, small molecules, oligonucleotides (including short interfering RNAs, RNAs and aptamers), peptides, polypeptides (including aptamers, zinc fingers and fragments thereof), proteins (including antibodies and fragments thereof) as well as derivatives or modified forms thereof.

It is readily apparent to one skilled in the art that this described method can be applied to a neuronal cell line, neuronal progenitor cells, cells collected from a patient with ALS, FTD, or both, who is a carrier of a C9ORF72 hexanucleotide expansion, or cells derived from cells collected from a human subject such as induced pluripotent stem cells derived from fibroblasts or plasma cells. It is readily apparent that this assay can be performed using blood, plasma, serum, cerebral spinal fluid or isolated blood cells collected from human subjects carrying a C9ORF72 hexanucleotide expansion, and can be used to diagnose ALS, FTD, or both, in these subjects. It is further apparent to one skilled in the art that this assay can be performed with human embryonic kidney cells, fibroblasts, lymphocytes or other cells commonly used in drug discovery and development.

Determination of the activity of C9ORF72-mediated genes in a biochemical assay: A method for measuring modulation by a molecule of the activity of an C9ORF72-mediated gene expression product can comprise a biochemical assay. A biochemical assay is substantially devoid of whole cells but may contain cell extracts or other cellular components.

The C9ORF72-mediated gene expression product can be immobilized on a solid support and the molecule is brought into contact with it in a reaction chamber. The binding of the molecule is quantified in an appropriate instrument using techniques known to one skilled in the art. In a preferred embodiment, the binding is quantified by surface plasmon resonance technology. Alternatively, the molecule is immobilized to the solid support and the C9ORF72-mediated gene expression product is brought into proximity in an appropriate reaction chamber. The C9ORF72-mediated gene expression product can be a recombinantly expressed, or a fragment thereof.

The biochemical assay can comprise an electrophoretic mobility shift assay (EMSA). An oligonucleotide including but not limited to one containing the SEQ ID NO: 1 is labeled with a radioactive, fluorescent or biotin label and incubated with a transcription factor to allow complex formation. In one embodiment, the transcription factor is recombinantly expressed, or a fragment thereof. In another embodiment, the transcription factor is chemically synthesized or recombinantly expressed SP1, KLF5, ZFX, KLF4, SP2, EGR1, KLF16 or TFAP2A or a fragment thereof. In another embodiment, SP1, KLF5, ZFX, KLF4, SP2, EGR1, KLF16 or TFAP2A, or a fragment thereof is contained in a nuclear extract of a cell. In one embodiment the oligonucleotide is 11 to 30 nucleotides in length comprising 11 to 24 consecutive nucleotides within SEQ ID NO:1 The reaction mixture is then analyzed by gel electrophoresis. If SP1, KLF5, ZFX, KLF4, SP2, EGR1, KLF16 or TFAP2A, or a fragment thereof is bound to the oligonucleotide it shifts the observed mobility of the labeled oligonucleotide towards an apparent higher molecular weight.

In a further embodiment, the biochemical assays can comprise surface plasmon resonance. Instruments employing surface plasmon resonance are known to the one skilled in the art. In a typical surface plasmon resonance assay, an oligonucleotide including but not limited to one containing the SEQ ID NO: 1 is immobilized on a chip which is a component of the surface plasmon resonance detection instrument. A solution containing a chemically synthesized or recombinantly expressed transcription factor SP1, KLF5, ZFX, KLF4, SP2, EGR1, KLF16 or TFAP2A or a fragment thereof is contacted with the oligonucleotide immobilized on the chip and surface plasmon resonance is used to detect binding of the oligonucleotide to one of said transcription factors. In another embodiment, the same technology is applied but the chemically synthesized or recombinantly expressed transcription factor SP1, KLF5, ZFX, KLF4, SP2, EGR1, KLF16 or TFAP2A or a fragment thereof is immobilized on the chip and a solution containing the oligonucleotide is contacted with the immobilized transcription factor.

The invention also includes oligonucleotides targeting any transcript of an C9ORF72-mediated gene. Such oligonucleotide may comprise an oligonucleotide of 11 to 30 nucleotides in length which binds to the transcription product of a C9ORF72-mediated gene. It may be a single-strand oligonucleotide or a double-strand oligonucleotide. For use as a therapeutic agent, such oligonucleotide may further comprise a pharmaceutically acceptable carrier. Optionally it may be chemically modified or formulated to enable transport into the brain across the blood-brain barrier. Preferred modifications include a phosphorothioate oligonucleotide, a methylphosphonate oligonucleotide, a phosphoamidite oligonucleotide, a peptide nucleic acid oligonucleotide, a locked-nucleic acid-modified oligonucleotide, and combinations thereof. The invention includes methods for modulating expression of a C9ORF72-mediated gene in a cell, said method comprising contacting a cell with an oligonucleotide disclosed herein.

In another embodiment, the oligonucleotide is fused to a cell penetrating peptide.

In another embodiment, the oligonucleotide is a short-interfering RNA (siRNA). There are several methods for preparing siRNA, such as chemical synthesis, in vitro transcription, siRNA expression vectors, and PCR expression cassettes. Irrespective of which method one uses, the first step in designing a siRNA requires choosing the siRNA target site. Standard guidelines for choosing siRNA target sites available in the current literature. Using standard guidelines, approximately half of all siRNAs yield >50% reduction in target mRNA levels, thus providing molecules potentially useful in the treatment of ALS, FTD, or both, in carriers of a C9ORF72 hexanucleotide expansion and of great interest for further investigation and secondary screening.

Antibodies and antigen binding fragments thereof. The term “antibody” as used to herein can include whole antibodies and refers, in one embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. In certain naturally occurring IgG, IgD, and IgA antibodies, the heavy chain constant region is comprised of three domains, CH1, CH2 and CH3. In certain naturally occurring antibodies, each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

Antibodies typically bind specifically to their cognate antigen with high affinity, reflected by a dissociation constant (K_{D}) of 10⁻⁷ to 10⁻¹¹ M or less. Any K_{D} greater than about 10⁻⁶ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸ M or less, even more preferably 5×10⁻⁹ M or less, and most preferably between 10⁻⁸ M and 10⁻¹⁰ M or less, but does not bind with high affinity to unrelated antigens.

Antibodies can also include, by way of example, both naturally occurring and non-naturally occurring antibodies; monoclonal and polyclonal antibodies; chimeric and humanized antibodies; fully-human antibodies and many variations known in the art.

The phrase “selectively binds to” refers to the ability of an antibody, antigen binding fragment or binding partner (antigen binding peptide) to preferentially bind to a C9ORF72-mediated gene expression product. Often the phrase “selectively binds” refers to the specific binding of antibody, fragment thereof, or binding partner to an antigen. The level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

Isolated antibodies of the invention can include serum containing such antibodies, or antibodies that have been purified to varying degrees. Whole antibodies of the invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), can also be employed in the invention.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies can be produced according to the methodology of Kohler and Milstein (Nature (1975), 256, 495-497). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

The term “antigen-binding fragment” of an antibody, as used herein, refers to one or more fragments of an antibody that retain the ability to selectively bind to an antigen (e.g., the expression product of a C9ORF72-mediated gene). It has been shown that the antigen-binding function of an antibody can be performed by fragments of a full-length antibody. Examples of binding fragments encompassed within the term “antigen-binding fragment” of an antibody, e.g., an antibody directed to the expression product of a C9ORF72-mediated gene, include (i) a Fab fragment, a monovalent fragment consisting of the V_{L}, V_{H}, CL and CH1 domains; (ii) a F(ab′) 2 fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) or (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These and other potential constructs are described at Chan & Carter (2010) Nat. Rev. Immunol. 10:301. Those skilled in the art are also familiar with antigen-binding fragments such as minibodies, cys-diabodies and fibronectin binding domains, which are also included in the invention herein. These antigen binding fragments are obtained using conventional techniques known to those with skill in the art, and the fragments are screened for utility in the same manner as are intact antibodies. Antigen-binding fragments can be produced by recombinant DNA techniques, or by enzymatic or chemical cleavage of intact immunoglobulins.

A “bispecific” or “bifunctional antibody” is an artificial hybrid antibody having two different heavy/light chain pairs and two different binding sites. Bispecific antibodies can be produced by a variety of methods including fusion of hybridomas, or by linking of antigen-binding fragments. See, e.g., Songsivilai & Lachmann, Clin. Exp. Immunol. 79:315-321 (1990); Kostelny et al., J. Immunol. 148, 1547-1553 (1992). The invention includes a bispecific antibody for use in the treatment of ALS, FTD, or both, when one or both of the binding sites are specific for the expression product of a C9ORF72-mediated gene. Based on the invention disclosed herein, an attractive avenue for therapeutic antibodies and antigen-binding fragments thereof includes modifications to enhance transport from the blood to the brain across the blood-brain barrier (BBB).

Antibodies have demonstrated the capacity to cross the blood-brain barrier (“BBB”) on their own, often in cases of BBB defects (Prins and Scheltens (2013) Alzheimer's Research & Therapy 5:56; Doody et al. N Engl J Med 370:4). Antibody fragments able to do so have been isolated by phenotypic panning of a naive llama single-domain antibody phage display library. Single domain antibodies, also referred to as nanobodies, are derived from camelids, which make a unique subset of immunoglobulins consisting of heavy chain homodimers devoid of light chains. Their variable region (VHH) is the smallest antigen-binding single polypeptide chain naturally found in the antibody world. Selected antibodies FC5 and FC44 demonstrated significantly (p<0.01) enhanced transport (50-100-fold) across the BBB in a rat in vitro model compared to control VHHs.

An alternative to enhance BBB transport is to employ linker molecules that transport antibodies/fragments from the blood into the brain. For example, specific brain delivery is achieved by engineering bispecific antibodies in which a therapeutic “arm” is combined with a BBB-transcytosing arm. Such work is based on recognized BBB specific receptors and transporters. Many endogenous molecules in circulation are able to cross the BBB via specific receptors and transporters expressed on the luminal side of brain endothelial cells, a process known as receptor-mediated transcytosis. Antibodies generated against these receptors, e.g. transferrin receptor (TFRC), insulin receptor (INSR), low density lipoprotein receptor-related protein 1 (LRP1), Basigin (Ok Blood Group, BSG), Glucose Transporter Type 1 (SLC2A1) and solute carrier CD98hc (SLC3A2) have been shown to accumulate in the brain in vivo. These antibodies can be used as platform to deliver therapeutic antibodies across the BBB (Bispecific antibodies in which one half targets the transport system, and the other half is the therapeutic antibody). Bispecific antibodies against the transferrin receptor and BACEI have been shown to traverse the blood-brain barrier and effectively reduce brain amyloid β3 levels.

The single-domain antibody FC5 has been shown to engage an active receptor mediated transport process by binding a putative a (2,3)-sialoglycoprotein receptor. Use of FC5 as the BBB-carrier arm in bispecific antibodies or antibody-drug conjugates offers an avenue to develop pharmacologically active biotherapeutics for CNS indications.

Another BBB transport moiety is the heavy-chain only antibodies identified in sharks. Antigen binding is mediated by a small and highly stable domain, known as VNAR. Antigen-specific VNAR molecules have been generated against a multitude of different targets via immunization, for instance VNAR that target the BBB transferrin receptor, as demonstrated by Ossianix Inc (Philadelphia, PA). This VNAR can be incorporated into a bispecific antibody, wherein the other binding moiety targets the expression product of a C9ORF72-mediated gene.

Diagnosis and prognosis of ALS, FTD, or both, using C9ORF72-mediated genes: Another aspect of the present invention provides diagnostic assays for measuring levels of an C9ORF72-mediated gene, or its protein activity, in the context of a biological sample (e.g., blood, urine, biopsies, lymph, saliva, cerebral spinal fluid) to thereby contribute to diagnosis of ALS, FTD, or both, in a carrier of a C9ORF72 hexanucleotide expansion.

Tissues, cells or body fluids from subjects carrying a C9ORF72 hexanucleotide expansion are collected and analyzed for expression levels of a C9ORF72-mediated gene listed in Table 1. There are numerous methods known to the skilled artisan to measure protein or mRNA expression levels in tissues, cells or body fluids. In a preferred embodiment, the tissue collected from subjects for expression analysis includes whole blood. In another preferred embodiment, fluids collected from subjects for expression analysis include blood plasma, blood serum, sputum, saliva or cerebrospinal fluid. In yet another preferred embodiment, cells collected from subjects include blood cells, buccal cells or skin fibroblasts.

An exemplary method for detecting the presence or absence of C9ORF72-mediated protein or nucleic acid in a biological sample involves obtaining a biological sample from a test subject and contacting the biological sample with a compound or an agent capable of detecting the protein or a nucleic acid (e.g., mRNA) that encodes the protein such that the presence of the protein or nucleic acid is detected in the biological sample. A preferred agent for detecting mRNA is a labeled nucleic acid probe capable of hybridizing to the mRNA. The nucleic acid probe can be, for example, a nucleic acid or a corresponding nucleic acid for a C9ORF72-mediated gene such as an oligonucleotide of at least 15, 30, 50, 100, 250 or 500 nucleotides in length which is capable of specifically hybridizing under stringent conditions to the mRNA. Other suitable probes for use in the diagnostic assays of the invention are known to those skilled in the art.

A preferred agent for detecting protein expression is an antibody capable of binding to a protein expressed from an C9ORF72-mediated gene, preferably an antibody with a detectable label. Antibodies can be polyclonal, or more preferably, monoclonal. An intact antibody, or a fragment thereof (e.g., Fab or F(ab′)2) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

With respect to antibody-based detection techniques, one of skill in the art can raise antibodies against an appropriate immunogen of C9ORF72-mediated gene protein expression product using no more than routine experimentation. Conditions for using such antibodies in a diagnostic assay include incubating an antibody with a test sample under conditions that vary depending upon the tissue or cellular type. Incubation conditions can depend on the format employed in the assay, the detection methods employed, and the type and nature of the antibody used in the assay. One skilled in the art will recognize that any one of the commonly available immunological assay formats (such as radioimmunoassays, enzyme-linked immunosorbent assays, diffusion based Ouchterlony, or rocket immunofluorescent assays) can readily be adapted to employ an antibody of the invention. Examples of such assays can be found in Chard, “An Introduction to Radioimmunoassay and Related Techniques,” Elsevier Science Publishers, Amsterdam, The Netherlands (1986); Bullock et al., “Techniques in Immunocytochemistry,” Academic Press, Orlando, Fla. Vol. 1 (1982), Vol. 2 (1983), Vol. 3 (1985); Tijssen, “Practice and Theory of enzyme Immunoassays: Laboratory Techniques in Biochemistry and Molecular Biology,” is Elsevier Science Publishers, Amsterdam, The Netherlands (1985).

The diagnostic methods of the invention can be used to detect mRNA or protein of a C9ORF72-mediated gene in a biological sample in vitro as well as in vivo. For example, in vitro techniques for detection of mRNA include northern blot hybridizations and in situ hybridizations. In vitro techniques for detection of proteins include enzyme linked immunosorbent assays (ELISAs), Western blots, immunoprecipitations, immunofluorescence, or quantitative sequencing reactions. Protein or mRNA levels can also be measured in an assay designed to evaluate a panel of target genes, e.g., a microarray or multiplex sequencing reaction.

The invention also provides kits for detecting the presence of a C9ORF72-mediated gene transcript or its protein expression product in a biological sample. For example, the kit can comprise a labeled compound or agent capable of detecting such protein or mRNA in a biological sample; means for determining the amount of such protein or mRNA in the sample; and means for comparing the amount in the sample with a known standard. The compound or agent can be packaged in a suitable container. The kit can further comprise instructions for using the kit.

One of skill in the art would be capable of performing these well-established protocols for the methods of the invention. (See, for example, Ausubel, et al., “Current Protocols in Molecular Biology,” John Wiley & Sons, NY, N. Y. (1999)).

Companion diagnostic or selection of therapeutic agent: The diagnostic methods of the invention provide advantages in the selection of an appropriate therapeutic agent for treatment or prevention of ALS, FTD, or both, in a carrier of a C9ORF72 hexanucleotide expansion. Given that a variety of therapeutic agents are employed in the treatment of symptoms of ALS, FTD, or both, in a carrier of a C9ORF72 hexanucleotide expansion and new therapeutics are anticipated in coming years, it is now possible to stratify patients who are responders or non-responders to such treatments on the basis of the activity or expression of the C9ORF72-mediated genes. Those of skill in the art can now perform clinical trials which correlate drug responsiveness with the results of one or more diagnostic assays for C9ORF72-mediated genes and their protein products.

At least two categories of such tests are immediately apparent. In one, a method for selecting a therapeutic agent for administration to a subject carrying a C9ORF72 hexanucleotide expansion having, or at-risk of having ALS, FTD, or both, comprises measuring the activity of an C9ORF72-mediated gene in a biological sample from the subject, and selecting a therapeutic agent based on whether the subject demonstrates differentiated activity of said gene in said sample. This example facilitates selection of a therapeutic agent of any type for the patient based on the diagnostic result.

In another method, a therapeutic agent which modulates the activity of an C9ORF72-mediated gene expression product may be recommended only if the subject who carries a C9ORF72 hexanucleotide expansion demonstrates a differentiated level of such gene expression product in the tissue sample. In this case the diagnostic test for an C9ORF72-mediated gene expression product is commonly known as a companion diagnostic. In such cases, if said subject does not demonstrate a differentiated level of gene or protein activity, the treatment is different than if he/she does.

### Experimental Validation of (9ORF72-Mediated Genes as Diagnostic and Therapeutic Targets in Human Neuronal Cells

Differentiated motor neurons carrying the C9ORF72 hexanucleotide expansion or the C9ORF72 wild-type allele can be procured by one of ordinary skill in the art. The differentiated motor neurons served as the tissue source to isolate the starting genetic material necessary for the differential gene expression analysis using qPCR. The differential gene expression analysis determined the relative expression levels of C9ORF72-mediated genes in motor neurons with the C9ORF72 hexanucleotide expansion relative to the expression levels in motor neurons with the C9ORF72 wild type. Total RNA was extracted using the Direct-zol™ RNA MiniPrep Kit (Zymo Research, Irvine, CA; Cat. no. R2050) according to the manufacturer's instructions with optional on-column DNase treatment. Total RNA was extracted from 5.0×10⁶ C9ORF72 hexanucleotide expansion neurons and 5.0×10⁶ C9ORF72 wild-type neurons. RNA concentration and purity were determined using a Nanodrop ND-1000 spectrophotometer (Thermo Scientific). RNA integrity was measured using the Agilent 2100 Bioanalyzer (Agilent Technologies, Palo Alto, CA) and the RNA 6000 Nano Chip Kit according to the manufacturer's instructions. Subsequently, 3 μg total RNA was used as template to synthesize cDNA (complementary deoxyribonucleic acid) with the High Capacity cDNA Reverse Transcription Kit (Applied Biosystems, Foster City, CA; Cat. no. 4368814). Detection of PCR products was facilitated by the use of a fluorescent reporter molecule in the reaction that yields increased fluorescence with an increasing amount of product DNA. A method of detection was employed that involves the double-stranded DNA intercalating molecule SYBR Green® to determine gene expression levels of protein encoding genes located within 2 Mb of C9ORF72. Real time PCR was performed on the BioRad CFX384 Real Time System (BioRad, Hercules, CA) using the panel of C9ORF72-mediated genes. Each reaction well contained 5 μL of PowerUp™ SYBR Green Master Mix (Applied Biosystems; Cat. no. A25742), cDNA equivalent to 13 ng of total RNA and 250 nM each of forward and reverse amplification primers in a final reaction volume of 10 μL. Cycling conditions were as follows: 95° C. for 10 minutes for polymerase activation, followed by 40 cycles of 95° C. for 15 seconds and 60° C. for 1 minute. Ribosomal protein L13 (RPL13) was used as a reference gene. Each quantitative qPCR experiment including amplification and data reduction was performed in triplicate. Data analysis was performed using CFX Manager software from BioRad, version 3.1. The experimental Cq (cycle quantification) was calibrated against the endogenous control products. Quantification of nucleic acids was achieved using relative quantification analysis (Double delta Ct data analysis, ΔΔCt method). Relative quantification analysis allowed to determine fold-change in expression of the target gene in the test cell line relative to the control cell line. First, a baseline was subtracted from the raw data based on the raw fluorescence values. Next, the threshold cycle (Ct) value was determined for each sample, which represents the number of cycles needed to reach a particular quantification threshold fluorescence signal level. Ct values were determined both for the genes being evaluated and for reference genes for normalization purposes. Average Ct values were determined for a gene of interest and the reference genes (designated as “Ref”) in the C9ORF72 hexanucleotide expansion neurons and C9ORF72 wild-type control neurons. For further data analysis, the C9ORF72 hexanucleotide expansion neuron samples were compared to the C9ORF72 wild-type control cell samples. For these paired comparisons, the following four values were generated: Avg. Ct Ref in C9ORF72 hexanucleotide expansion, Avg. Ct Ref in C9ORF72 wild-type, Avg. Ct gene of interest in C9ORF72 hexanucleotide expansion, and Avg. Ct gene of interest in C9ORF72 wild-type. The differences between Ct values of the gene of interest and reference genes (delta Ct values, short dCt) were calculated for the C9ORF72 hexanucleotide expansion and the C9ORF72 wild-type control. Next, the difference between C90RF72 hexanucleotide expansion and C9ORF72 wild-type were calculated to arrive at the Double Delta Ct Value (ddCt C9ORF72 hexanucleotide expansion-C9ORF72 wild-type). Since the quantity of amplified product doubles in each cycle, the expression fold-change between C9ORF72 hexanucleotide expansion and C9ORF72 wild-type control (Relative Quantification or RQ (C9ORF72 hexanucleotide expansion/C9ORF72 wild-type)) was computed using the following formula 2\hat{ }−^{ddCt}.

TABLE 3
Relative expression levels of C9ORF72 mediated genes located
within a 2 Mb range of C9ORF72 on human chromosome 9. Expression
levels in motorneurons with C9ORF72 wild-type were compared
to expression levels in motorneurons containing a C9ORF72
hexanucleotide expansion. Fold-change means the relative
expression level of a gene in C9ORF72 hexanucleotide expansion
neurons compared to C9ORF72 wild-type neurons. Negative
values indicate downregulation in C9ORF72 hexanucleotide
expansion neurons compared to C9ORF72 wild-type neurons,
positive values indicate upregulation.

| | C9ORF72 hexanucleotide expansion |
|---|---|
| C9ORF72- | versus C9ORF72 wild-type |
| mediated gene | mRNA expression (Fold-change) |
| CAAP1 | −1.259 |
| EQTN | −5.503 |
| IFNK | −1.009 |
| IFT74 | 1.529 |
| IZUMO3 | 3.515 |
| LINGO2 | −1.183 |
| LRRC19 | 1.123 |
| MOB3B | −3.267 |
| PLAA | −1.344 |
| TEK | 3.506 |
| TUSC1 | −1.232 |

Relative expression levels of C9ORF72-mediated genes were determined for CAAP1, EQTN, IFNK, IFT74, IZUMO3, LINGO2, LRRC19, MOB3B, PLAA, TEK and TUSC1. A strong downregulation of expression in C9ORF72 hexanucleotide expansion neurons compared to C9ORF72 wild-type neurons was determined for EQTN and MOB3B. A strong upregulation of expression in C9ORF72 hexanucleotide expansion neurons compared to C9ORF72 wild-type neurons was determined for IZUMO3 and TEK.

Identification of binding motifs for the transcription factors in the 5′-sequences of C9ORF72-mediated genes. A JASPAR analysis of 5′-sequences for the genes strongly up- or downregulated was completed as functional transcription factor binding sites are likely to be observed between 100 bp downstream and about 200 bp upstream from the transcription start site but can sometimes be found further away. A relative profile score threshold cut-off of 85% was used. The JASPAR analysis showed that the 5′ DNA region of the C9ORF72-mediated gene MOB3B contains high scoring binding motifs (>90% relative score) for transcription factors EGR1, KLF5, SP1 and TFAP2A. The 5′ DNA region of the C9ORF72-mediated gene EQTN contains high scoring binding motifs (>90% relative score) for transcription factors EGR1, KLF4, KLF5, KLF16, SP1 and TFAP2A. The 5′ DNA region of the C9ORF72-mediated gene IZUMO3 contains high scoring binding motifs (>90% relative score) for transcription factor KLF5. The 5′ DNA region of the C9ORF72-mediated gene TEK contains high scoring binding motifs (>90% relative score) for transcription factors KLF4 and KLF5. Table 4 shows the highest Relative Score obtained for above mentioned transcription factors for these four genes.

TABLE 4
JASPAR analysis of selected transcription factors'
binding sites in the 5′ DNA region of C9ORF72-mediated
genes MOB3B, EQTN, IZUMO3 and TEK.

| | | Gene | | | |
|---|---|---|---|---|---|
| | | MOB3B | EQTN | IZUMO3 | TEK |
| Model ID | Model name | Highest Relative Score | | | |
| MA0079.3 | EGR1 | 0.9136 | 0.9712 | | |
| MA0599.1 | KLF16 | | 0.9257 | | |
| MA0146.2 | KLF4 | | 0.9311 | | 0.9427 |
| MA0039.2 | KLF5 | 0.9196 | 0.9755 | 0.9179 | 0.9505 |
| MA0516.1 | SP1 | 0.9112 | 0.9623 | | |
| MA0741.1 | TFAP2A | 0.9230 | 0.9261 | | |

Together the examples herein provide experimental validation of C9ORF72-mediated genes and expression products thereof as targets for diagnosis, prevention and treatment of ALS, FTD, or both, in subjects carrying a C9ORF72 hexanucleotide expansion.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

### REFERENCES

ⁱ Taylor J P, Brown R H Jr, Cleveland D W. Decoding ALS: from genes to mechanism. Nature. 2016 Nov. 10; 539 (7628): 197-206. doi: 10.1038/nature20413. ⁱⁱ Mejzini R, Flynn L L, Pitout I L, Fletcher S, Wilton S D, Akkari P A. ALS Genetics, Mechanisms, and Therapeutics: Where Are We Now? Front Neurosci. 2019 Dec. 6:13:1310. doi: 10.3389/fnins.2019.01310. eCollection 2019.
ⁱⁱⁱ Gamez J, Corbera-Bellalta M, Nogales G, Raguer N, Garcia-Arumi E, Badia-Canto M, Lladó-Carbó E, Alvarez-Sabin J. Mutational analysis of the Cu/Zn superoxide dismutase gene in a Catalan ALS population: should all sporadic ALS cases also be screened for SOD1? J Neurol Sci. 2006 Aug. 15; 247 (1): 21-8. Epub 2006 May 3.
^{iv} Cooper-Knock J, Hewitt C, Highley J R, Brockington A, Milano A, Man S, Martindale J, Hartley J, Walsh T, Gelsthorpe C, Baxter L, Forster G, Fox M, Bury J, Mok K, McDermott C J, Traynor B J, Kirby J, Wharton S B, Ince P G, Hardy J, Shaw P J. Clinico-pathological features in amyotrophic lateral sclerosis with expansions in C9ORF72. Brain. 2012 March: 135 (Pt 3): 751-64. doi: 10.1093/brain/awr365.
^{v} Graff-Radford N R, Woodruff B K. Frontotemporal dementia. Semin Neurol. 2007 February: 27 (1): 48-57.
^{vi} Coyle-Gilchrist I T, Dick K M, Patterson K, Vazquez Rodriquez P, Wehmann E, Wilcox A, Lansdall C J, Dawson K E, Wiggins J, Mead S, Brayne C, Rowe J B. Prevalence, characteristics, and survival of frontotemporal lobar degeneration syndromes. Neurology. 2016 May 3; 86 (18): 1736-43. doi: 10.1212/WNL.0000000000002638. Epub 2016 Apr. 1.
^{vii} Lomen-Hoerth C, Anderson T, Miller B. The overlap of amyotrophic lateral sclerosis and frontotemporal dementia. Neurology. 2002 Oct. 8; 59 (7): 1077-9.
^{viii} Panza F, Lozupone M, Seripa D, Daniele A, Watling M, Giannelli G, Imbimbo B P. Development of disease-modifying drugs for frontotemporal dementia spectrum disorders. Nat Rev Neurol. 2020 April: 16 (4): 213-228. doi: 10.1038/s41582-020-0330-x. Epub 2020 Mar. 23.
^{ix} Greaves C V, Rohrer J D. An update on genetic frontotemporal dementia. J Neurol. 2019 August: 266 (8): 2075-2086. doi: 10.1007/s00415-019-09363-4. Epub 2019 May 22.
^{x} DeJesus-Hernandez M, Mackenzie I R, Boeve B F, Boxer A L, Baker M, Rutherford N J, Nicholson A M, Finch N A, Flynn H, Adamson J, Kouri N, Wojtas A, Sengdy P, Hsiung G Y, Karydas A, Seeley W W, Josephs K A, Coppola G, Geschwind D H, Wszolek Z K, Feldman H, Knopman D S, Petersen R C, Miller B L, Dickson D W, Boylan K B, Graff-Radford N R, Rademakers R. Expanded GGGGCC hexanucleotide repeat in noncoding region of C9ORF72 causes chromosome 9p-linked FTD and ALS. Neuron. 2011 Oct. 20; 72 (2): 245-56. doi: 10.1016/j.neuron.2011.09.011. Epub 2011 Sep. 21.
^{xi} Renton A E, Majounie E, Waite A, Simon-Sánchez J, Rollinson S, Gibbs J R, Schymick J C, Laaksovirta H, van Swieten J C, Myllykangas L, Kalimo H, Paetau A, Abramzon Y, Remes A M, Kaganovich A, Scholz S W, Duckworth J, Ding J, Harmer D W, Hernandez D G, Johnson J O, Mok K, Ryten M, Trabzuni D, Guerreiro R J, Orrell R W, Neal J, Murray A, Pearson J, Jansen I E, Sondervan D, Seelaar H, Blake D, Young K, Halliwell N, Callister J B, Toulson G, Richardson A, Gerhard A, Snowden J, Mann D, Neary D, Nalls M A, Peuralinna T, Jansson L, Isoviita V M, Kaivorinne A L, Hölttä-Vuori M, Ikonen E, Sulkava R, Benatar M, Wuu J, Chiò A, Restagno G, Borghero G, Sabatelli M: ITALSGEN Consortium, Heckerman D, Rogaeva E, Zinman L, Rothstein J D, Sendtner M, Drepper C, Eichler E E, Alkan C, Abdullaev Z, Pack S D, Dutra A, Pak E, Hardy J, Singleton A, Williams N M, Heutink P, Pickering-Brown S, Morris H R, Tienari P J, Traynor B J. A hexanucleotide repeat expansion in C9ORF72 is the cause of chromosome 9p21-linked ALS-FTD. Neuron. 2011 Oct. 20; 72 (2): 257-68. doi: 10.1016/j.neuron.2011.09.010. Epub 2011 Sep. 21.
^{xii} Rao S S, Huntley M H, Durand N C, Stamenova E K, Bochkov I D, Robinson J T, Sanborn A L, Machol I, Omer A D, Lander E S, Aiden E L. A 3D map of the human genome at kilobase resolution reveals principles of chromatin looping. Cell. 2014 Dec. 18; 159 (7): 1665-80. doi: 10.1016/j.cell.2014.11.021. Epub 2014 Dec. 11. Erratum in: Cell. 2015 Jul. 30; 162 (3): 687-8.
^{xiii} Benabdallah N S, Bickmore W A. Regulatory Domains and Their Mechanisms. Cold Spring Harb Symp Quant Biol. 2015:80:45-51. doi: 10.1101/sqb.2015.80.027268. Epub 2015 Nov. 20. Review.
^{xiv} Miele A, Dekker J. Long-range chromosomal interactions and gene regulation. Mol Biosyst. 2008 November: 4 (11): 1046-57. doi: 10.1039/b803580f. Epub 2008 Aug. 13.
^{xv} Erceg J, Saunders T E, Girardot C, Devos D P, Hufnagel L, Furlong E E. Subtle changes in motif positioning cause tissue-specific effects on robustness of an enhancer's activity. PLOS Genet. 2014 January: 10 (1): e 1004060. doi: 10.1371/journal.pgen. 1004060. Epub 2014 Jan. 2.
^{xvi} Rouillard A D, Gundersen G W, Fernandez N F, Wang Z, Monteiro C D, McDermott M G, Ma′ayan A. The harmonizome: a collection of processed datasets gathered to serve and mine knowledge about genes and proteins. Database (Oxford). 2016 Jul. 3:2016. pii: baw 100.
^{xvii} Yu F X, Guan K L. The Hippo pathway: regulators and regulations. Genes Dev. 2013 Feb. 15; 27 (4): 355-71.
^{xviii} Mana-Capelli S, Paramasivam M, Dutta S, McCollum D. Angiomotins link F-actin architecture to Hippo pathway signaling. Mol Biol Cell. 2014 May: 25 (10): 1676-85.
^{xix} Serrano I. McDonald P C, Lock F, Muller W J, Dedhar S. Inactivation of the Hippo tumour suppressor pathway by integrin-linked kinase. Nat Commun. 2013:4:2976. doi: 10.1038/ncomms3976.
^{xx} Nardone G, Oliver-De La Cruz J, Vrbsky J, Martini C, Pribyl J, Skládal P, Pešl M, Caluori G, Pagliari S, Martino F, Maceckova Z, Hajduch M, Sanz-Garcia A, Pugno N M, Stokin G B, Forte G. YAP regulates cell mechanics by controlling focal adhesion assembly. Nat Commun. 2017 May 15; 8:15321.
^{xxi} Qiao Y, Chen J, Lim Y B, Finch-Edmondson M L, Seshachalam V P, Qin L, Jiang T, Low B C, Singh H, Lim C T, Sudol M. YAP Regulates Actin Dynamics through ARHGAP29 and Promotes Metastasis. Cell Rep. 2017 May 23; 19 (8): 1495-1502.
^{xxii} Tao S C, Gao Y S, Zhu H Y, Yin J H, Chen Y X, Zhang Y L, Guo S C, Zhang C Q. Decreased extracellular pH inhibits osteogenesis through proton-sensing GPR4-mediated suppression of yes-associated protein. Sci Rep. 2016 Jun. 3; 6:26835.
^{xxiii} Komuro A, Nagai M, Navin N E, Sudol M. WW domain-containing protein YAP associates with ErbB4 and acts as a co-transcriptional activator for the carboxyl-terminal fragment of ErbB-4 that translocates to the nucleus. J Biol Chem. 2003 Aug. 29; 278 (35): 33334-41. Epub 2003 Jun. 13.
^{xxiv} Rouillard A D, Gundersen G W, Fernandez N F, Wang Z, Monteiro C D, McDermott M G, Ma′ayan A. The harmonizome: a collection of processed datasets gathered to serve and mine knowledge about genes and proteins. Database (Oxford). 2016 Jul. 3:2016. pii: baw 100.
^{xxv} W Zhang Y, Roland C, Sagui C. Structural and Dynamical Characterization of DNA and RNA Quadruplexes Obtained from the GGGGCC and GGGCCT Hexanucleotide Repeats Associated with C9FTD/ALS and SCA36 Diseases. ACS Chem Neurosci. 2018 May 16; 9 (5): 1104-1117. doi: 10.1021/acschemneuro.7b00476. Epub 2018 Jan. 12.
^{xxvi} Bidichandani S I, Ashizawa T, Patel P I. The GAA triplet-repeat expansion in Friedreich ataxia interferes with transcription and may be associated with an unusual DNA structure. Am J Hum Genet. 1998 January: 62 (1): 111-21.
^{xxvii} Cho D H, Thienes C P, Mahoney S E, Analau E, Filippova G N, Tapscott S J. Antisense transcription and heterochromatin at the DMI CTG repeats are constrained by CTCF. Mol Cell. 2005 Nov. 11; 20 (3): 483-9.
^{xxviii} Hu Z. Tee W W. Enhancers and chromatin structures: regulatory hubs in gene expression and diseases. Biosci Rep. 2017 Apr. 28; 37 (2): BSR20160183. doi: 10.1042/BSR20160183. PMID: 28351896: PMCID: PMC5408663.
^{xxix} Chen H, Li H. Liu F, Zheng X, Wang S, Bo X, Shu W. An integrative analysis of TFBS-clustered regions reveals new transcriptional regulation models on the accessible chromatin landscape. Sci Rep. 2015 Feb. 16:5:8465. doi: 10.1038/srep08465. PMID: 25682954; PMCID: PMC4329551.
^{xxx} van Dijk D, Sharon E, Lotan-Pompan M. Weinberger A, Segal E. Carey L B. Large-scale mapping of gene regulatory logic reveals context-dependent repression by transcriptional activators. Genome Res. 2017 January: 27 (1): 87-94. doi: 10.1101/gr.212316.116. Epub 2016 Dec. 13.
^{xxxi} Papadopoulos C, Kirchner P, Bug M, Grum D, Koerver L, Schulze N, Poehler R, Dressler A, Fengler S, Arhzaouy K, Lux V, Ehrmann M, Weihl C C, Meyer H. VCP/p97 cooperates with YOD1, UBXD1 and PLAA to drive clearance of ruptured lysosomes by autophagy. EMBO J. 2017 Jan. 17; 36 (2): 135-150. doi: 10.15252/embj.201695148. Epub 2016 Oct. 17.
^{xxxii} Seguin S J, Morelli F F, Vinet J, Amore D, De Biasi S, Poletti A, Rubinsztein D C, Carra S. Inhibition of autophagy, lysosome and VCP function impairs stress granule assembly. Cell Death Differ. 2014 December: 21 (12): 1838-51. doi: 10.1038/cdd.2014.103. Epub 2014 Jul. 18.
^{xxxiii} Avruch J. Zhou D, Fitamant J, Bardeesy N, Mou F, Barrufet L R. Protein kinases of the Hippo pathway: regulation and substrates. Semin Cell Dev Biol. 2012 September: 23 (7): 770-84. doi: 10.1016/j.semcdb.2012.07.002. Epub 2012 Aug. 9.
^{xxxiv} Chow A, Hao Y, Yang X. Molecular characterization of human homologs of yeast MOB1. Int J Cancer. 2010 May 1; 126 (9): 2079-89. doi: 10.1002/ijc.24878.
^{xxxv} Joung J, Engreitz J M, Konermann S, Abudayyeh O O, Verdine V K, Aguet F, Gootenberg J S, Sanjana N E, Wright J B, Fulco C P, Tseng Y Y, Yoon C H, Boehm J S, Lander E S, Zhang F. Genome-scale activation screen identifies a lncRNA locus regulating a gene neighbourhood. Nature. 2017 Aug. 17; 548 (7667): 343-346.
^{xxxvi} Kosacka J, Figiel M, Engele J, Hilbig H, Majewski M, Spanel-Borowski K. Angiopoietin-1 promotes neurite outgrowth from dorsal root ganglion cells positive for Tie-2 receptor. Cell Tissue Res. 2005 April: 320 (1): 11-9. Epub 2005 Feb. 16.
^{xxxvii} Lee H S, Oh S J, Lee K H, Lee Y S, Ko E, Kim K E, Kim H C, Kim S, Song P H, Kim Y I, Kim C, Han S. Gln-362 of angiopoietin-2 mediates migration of tumor and endothelial cells through association with a51 integrin. J Biol Chem. 2014 Nov. 7; 289 (45): 31330-40. doi: 10.1074/jbc.M114.572594. Epub 2014 Sep. 18.

1. A method for detecting a change in expression of at least one C9ORF72-mediated gene located on human chromosome 9 comprising isolating messenger RNA from a tissue or cell sample obtained from a carrier of a C9ORF72 hexanucleotide expansion suspected of having amyotrophic lateral sclerosis, frontotemporal dementia or both and synthesizing and amplifying cDNA of the C9ORF72-mediated gene from said tissue or cell sample and identifying a change in expression of the at least one C9ORF72-mediated gene, wherein the change in the expression of the at least one C9ORF72-mediated gene comprises a change in the amount of messenger RNA encoded by the at least one C9ORF72-mediated gene compared to an amount in messenger RNA isolated from a same tissue or cell sample from a healthy subject.

2. The method of claim 1, wherein the change in the expression of the at least one C9ORF72-mediated gene is detected in messenger RNA isolated from tissue or cell samples obtained from carriers of a C9ORF72 hexanucleotide expansion with ALS, FTD, or both.

3. The method of claim 1, wherein the at least one C9ORF72-mediated gene is located within about 2 Mb of the C9ORF72 gene.

4. The method of claim 1, wherein the at least one C9ORF72-mediated gene comprises at least one of IZUM03, TUSC1, CAAP1, PLAA, IFT74, LRRC19, TEK, EQTN, MOB3B, IFNK and LINGO2.

5. The method of claim 1, wherein the at least one C9ORF72-mediated gene comprises at least two of IZUM03, TUSC1, CAAP1, PLAA, IFT74, LRRC19, TEK, EQTN, MOB3B, IFNK and LINGO2.

6. The method of claim 1, wherein the at least one C9ORF72-mediated gene comprises at least three of IZUM03, TUSC1, CAAP1, PLAA, IFT74, LRRC19, TEK, EQTN, MOB3B, IFNK and LINGO2.

7. The method of claim 1, wherein the at least one C9ORF72-mediated gene comprises at least one of PLAA, MOB3B, TEK, IZUMO3 and EQTN.

8. (canceled)

9. The method of claim 1, wherein the expression of at least one C9ORF72-mediated gene is greater than its expression in messenger RNA isolated from the same tissue or cell sample obtained from a healthy subject.

10. The method of claim 9, wherein at least one C9ORF72-mediated gene comprises at least one of IFT74, IZUM03, LRRC19 and TEK.

11. The method of claim 1, wherein the expression of the least one C9ORF72-mediated gene is less than the expression of the C9ORF72-mediated gene in the amount of messenger RNA isolated from the same tissue or cell sample obtained from a healthy subject.

12. The method of claim 11, wherein the at least one C9ORF72-mediated gene comprises at least one of CAAP1, EQTN, IFNK, LINGO2, MOB3B, PLAA and TUSC1.

13. The method of claim 1, wherein the tissue or cell sample is whole blood, blood plasma, blood serum, sputum, saliva, urine, lymph or cerebrospinal fluid, human homozygous or heterozygous C9ORF72 hexanucleotide expansion carrying neurons, buccal cells, lymphocytes or skin fibroblasts.

14.-21. (canceled)

22. A cell-based assay for detecting a change in the expression of, or an activity of a gene expression product encoded by, a C9ORF72-mediated gene located on human chromosome 9.

23.-31. (canceled)

32. A kit for detecting a change in the expression of at least one C9ORF72-mediated gene in total RNA isolated from a sample obtained from a carrier of a C9ORF72 hexanucleotide expansion suspected of having ALS, FTD, or both, comprising an agent for detecting mRNA encoded by at least one C9ORF72-mediated gene comprising at least one of IZUM03, TUSC1, CAAP1, PLAA, IFT74, LRRC19, TEK, EQTN, MOB3B, IFNK and LINGO2.

33. The method according to claim 1 wherein an assay is used for detecting a change in the expression of at least one C9ORF72-mediated gene and the assay is a cell-based assay for detecting the change in the expression of, or the activity of a gene expression product encoded by, a C9ORF72-mediated gene located on human chromosome 9.

34. The method according to claim 23 wherein an assay is used for detecting a change in the expression of at least one C9ORF72-mediated gene and the assay is a cell-based assay for detecting a change in the expression of, or the activity of a gene expression product encoded by, a C9ORF72-mediated gene located on chromosome 9.

35. The method of claim 1, wherein the method comprises using a northern blot analysis or an in situ hybridization analysis.

36. The method of claim 2, wherein the method comprises using a northern blot analysis or an in situ hybridization analysis.

37. The method of claim 2, wherein the method comprises using a northern blot analysis or an in situ hybridization analysis

38. The kit of claim 32, wherein the kit employs a northern blot analysis or an in situ hybridization analysis.


SEQUENCE LISTING (1 sequence)

SEQ ID NO: 1 - DNA, 30 bp, Homo sapiens
  source 1..30: mol_type = genomic DNA
ggggccggggccggggccggggccggggcc